(19) United States
(12) Patent Application Publication
(10) Pub. No.: US 2006/0159666 A1
(43) Pub. Date: Jul. 20, 2006

(54) Method of potentiating inflammatory and immune modulation for cell and drug therapy

(76) Inventors: Alison E. Willing, Tampa, FL (US); Paul R. Sanberg, Spring Hill, FL (US); Mary B. Newman, Branden, FL (US); Cyndy Davis Sanberg, Spring Hill, FL (US)

Correspondence Address: THE LUTHER LAW FIRM, 12198 E. COLUMBINE DR., SCOTTSDALE, AZ, 85259, US

(21) Appl. No.: 11/258,290

(22) Filed: Oct. 24, 2005

Related U.S. Application Data
(60) Provisional application No. 60/621,341, filed on Oct. 22, 2004.

(51) Int. Cl. A61K 35/14

(52) U.S. Cl. 424/93.7

(57) ABSTRACT

A method for repairing animal tissue damage due to an inflammatory reaction in an animal has the steps of providing umbilical cord blood cells (UCBCs) in a pharmaceutically acceptable form; and administering a sufficient dose of UCBC at an optimal time thereby reducing the injury from the inflammatory reaction. Also provided are method of treating cerebrovascular accident, acute central nervous inflammation, multiple sclerosis, myocardial ischemia, and neonatal bronchopulmonary distress. For determining the optimal time of UCBCs administration, there is provided a kit containing antibodies for IL-8 and MCP-1.

### RELATED APPLICATIONS

This application claims the benefit of provisional application 69/69,341, filed Oct. 22, 2004, entitled Potentiation of Inflammatory and Immune Modulation for Cell and Drug Therapy, which is hereby incorporated by reference.

### TECHNICAL FIELD

The field of this invention is the treatment of various diseases and disorders using undifferentiated stem cells. In particular, the primary disorder is an ischemic event such as a cerebrovascular accident (CVA or stroke), and the stem cell is the human umbilical cord blood cell (HUCBC). More specifically, the HUCBC is injected systemically into an individual at a time interval that is sufficient to permit attraction of the stem cells to the site of injury and also allows for damaged and injured brain cells in the core area of injury to recover.

### BACKGROUND ART

Cerebrovascular accidents (CVAs), considered one of the top five non-communicable diseases, affect approximately 50 million people worldwide, resulting in approximately 5.5 million deaths per year. Of those 50 million worldwide, CVAs account for roughly 40 million people. CVA is the third leading cause of death in developed countries and accounts for the major cause of adult disability.

The CVA must be managed either by prevention or treatment. Prevention consists of primarily lifestyle and medical adjustments. Lifestyle changes include smoking cessation, regular exercise, nutritional modifications, including limiting sodium intake and moderating or stopping alcohol consumption. A common medical intervention is daily, low-dose aspirin therapy (commonly 81 mg/d of aspirin). Surgery appears to be effective for specific sub-groups. Angioplasty of cerebral arteries is still an experimental procedure with insufficient data for analysis. Other prophylactic medical adjustments include medications to lower blood pressure, lower cholesterol, control diabetes and control circulatory problems.

Acute treatments consist of the use of thrombolytics, neuroprotective agents, Oxygenated Fluorocarbon Nutrient Emulsion (OFNE) Therapy, neuroperfusion, GPIIb/IIIa platelet inhibitor therapy, and rehabilitation and physical therapy. A thrombolytic agent is intended to dissolve a blood clot or thrombosis (about 90% of CVAs). The most commonly used agent is recombinant tissue plasminogen activator (TPA; Alteplase, Genentech), but other thrombolytics also are available (e.g., streptokinase, Streptase from Aventis Behring; and urokinase, Abbokinase, Abbott Laboratories). The thrombolytic agent helps reestablish cerebral circulation by dissolving obstructive blood clots. While effective in some patients, it must be administered within a short time from formation of the blood clot. Importantly, the thrombolytic agent may cause expansion of the CVA volume due to additional hemorrhaging; therefore, prior to thrombolytic administration, an emergency CT scan is generally required, further reducing the time available.

There are a variety of proven and putative neuroprotective agents, including, for example, glutamate antagonists, calcium antagonists, opiate antagonists, GABA-A agonists, calpain inhibitors, kinase inhibitors and antioxidants. Several are undergoing clinical trials. Due to their complementary functions of thrombolysis and “brain protection,” future acute treatment procedures will most likely involve the combination of thrombolytic and neuroprotective therapies. However, like thrombolytics, most neuroprotective agents need to be administered within six hours or less after the onset of the CVA to be reasonably effective.

The OFNE procedure delivers oxygen and nutrients to the brain through the cerebral spinal fluid (CSF). Such neuroperfusion is an experimental procedure in which oxygen-rich fluid is rerouted through the brain as a way to minimize the damage of a CVA. GPIIb/IIIa platelet inhibitor therapy inhibits the ability of the glycoprotein GPIIb/IIIa receptors on platelets to aggregate, or clump, thus reducing formation of thrombolytic blood clots. Rehabilitation and physical therapy must begin soon after the CVA; however, this therapy is not known to change brain damage due to CVA. The goal of rehabilitation is to improve function so that the CVA survivor can become as functional and independent as possible.

Although some of the acute treatments showed promise in clinical trials, a study conducted in Cleveland, Ohio, showed that only 1.8% of patients presenting with CVA symptoms even received TPA (Katzen Ill., et al., 2000 JAMA, 283:1151-1158). TPA is currently the most effective and widely used of the above-mentioned acute CVA treatments; however, the number of patients receiving this acute CVA treatment is estimated to be under 10%. These statistics show a clear need for the availability of a subacute CVA treatment effective at more than a few hours after the CVA.

Most patients with a CVA deny or ignore symptoms for hours, only seeking treatment late in the progression of their acute event. Thus, “acute” therapies such as TPA are suboptimal at best, since most patients with CVAs first seek medical attention long after the acute period. Recent studies have shown that 42% of CVA patients wait as long as 24 hours before seeking treatment, with the average time of arrival being 13 hours after onset of the CVA. TPA has been shown to enhance recovery of about one third of the patients receiving that therapy; however, a recent study required by the FDA (Standard Treatment with Alteplase to Reverse Stroke) found that about a third of the time, the three-hour treatment window was violated, resulting in ineffective treatment. Similar results have been reported with other thrombolytics and platelet aggregation antagonists. With the exception of rehabilitation, the remaining acute treatments are still in clinical trials and are not widely available in the United States, particularly in rural areas, which lack large medical centers with the needed neurology specialists and emergency room staffing.

For rural areas, access to these new methods of CVA diagnosis and acute therapy may be limited for an extended period of time. The present invention allows such disadvantaged or rural patients to seek subsequent care within 48 hours.

The annual cost of CVA treatment in the US is over USD43 billion, including both direct and indirect costs. Direct costs account for about 60% of the total and include hospital stays, physicians' fees and rehabilitation. These costs normally reach USD 15,000 per patient in the first three months; however, in approximately 10% of the cases, the costs exceed USD35,000. Indirect costs account for 40% and include lost productivity of the CVA victim and family care givers.

About 750,000 CVAs occur in the USA every year, of which about one third are fatal. Of the remaining patients, one third of the CVAs are mildly impaired, one third of the CVAs are moderately impaired, and one third of the CVAs are severely impaired.

The risk of experiencing a CVA increases with age. As the baby-boomers age, the total number of CVAs likely will increase substantially. After 55, the risk of having a CVA doubles every decade, with approximately 40% of individuals over the age of 80 having had CVAs. Also the risk of having a second CVA increases over time. Within five years after a first CVA, the risk of having a second CVA is between 25% and 40%. With the over-65 population expected to increase with the aging of the baby-boomers, the size of this market will grow substantially. Also, the demand for an effective treatment will increase dramatically.

Given the current inability to effectively slow or mitigate the devastating effects of CVAs, it is imperative that novel therapeutic strategies are developed to both (1) minimize the initial CNS trauma and (2) repair the damaged brain.

Transplantation of stem cells has been proposed as a means of treating numerous diseases and conditions, including CVAs. The powerful multipotent potential of stem cells may make it possible to effectively treat diseases and injuries with complicated disruptions in neuronal physiology and function, such as CVAs, in which more than one cell type is affected. Neural stem cells are important treatment candidates for CVA and other CNS diseases because of their ability to differentiate in vitro and in vivo into neurons, astrocytes and oligodendrocytes.

Despite this great potential, an easily obtainable, abundant, safe and clinically proven source of stem cells has been elusive until recently. Umbilical cord blood contains a relatively high percentage of undifferentiated stem cells capable of differentiating into all of the major cellular phenotypes of the CNS, including neurons, oligodendrocytes, and glial cells (Sanchez-Ramos et al., 2001 Exp Neurol, 171(1):109-15; and Bicknese et al., 2002 Cell Transplant, 11(3):2612-4). Following intravenous delivery, human umbilical cord blood cells (HUCBC) survive and migrate into the CNS of diseased animals and have been shown to promote functional recovery in animal models of CVA, spinal cord injury, and hemorrhagic stroke (Chen et al., 2001 Stroke, 32(11):2682-8; Lu et al., 2002 Cell Transplant, 11(3):275-81; Saporta et al., 2003 J. Hematotherapy & Stem Cell Research, 12:271-278).

In addition to the growing body of evidence supporting the neurotherapeutic potential of HUCBCs, there is a long and well established series of practical advantages of using HUCBC for clinical diseases. Cord blood is easily obtained with no risks to the mother or child. A blood sample is taken from the umbilical vein attached to the placenta after birth. The percentage of the undifferentiated stem cells present in the mononuclear fraction is small; but the absolute yield of stem cells may number in the thousands prior to expansion or other ex vivo manipulation, providing an easily obtainable and plentiful source. Hematopoietic stem cells from HUCB have been routinely and safely used to reconstitute bone marrow and blood cell lineages in children with malignant and nonmalignant diseases after treatment with myeloablative doses of chemoradiotherapy (Lu et al., 1996 Crit Rev Oncol Hematol., 22(2):61-78; and Broxmeyer, Cellular Characteristics of cord blood and cord blood transplantation, In AABB Press. 1998 Bethesda, Md.). Early results indicate that a single cord blood sample provides enough hematopoietic stem cells to provide both short- and long-term engraftment. This suggests that these stem cells maintain extensive replicative capacity, which may not be true of hematopoietic stem cells obtained from other sources, such as adult bone marrow.

In addition, HUCBCs can also be easily cryopreserved following isolation. Cryopreservation of HUCBCs, accompanied by sustained good cell viability after thawing, also allows long-term storage and efficient shipment of cells from the laboratory to the clinic. Thus, this novel feature of cryopreservation gives HUCBCs a commercially distinct advantage in the design of cell-based therapeutic products. Although the duration of time that the cells may be stored with high viability upon thawing remains to be determined, it has been reported that after HUCBCs were frozen for at least 15 years, viable cells were thawed and survived transplant within animal models of injury (Broxmeyer et al., 2003 Proc Natl Acad Sci USA, 100(2):645-50).

Because HUCBC transplant recipients exhibit a low incidence and severity of graft-versus-host disease or immuno-rejection (Wagner et al., 1992 Blood, 79(7):1874-81; Gluckman et al., 1997 N Engl J Med., 337(6):373-81), long-term immune suppression with its associated health risks may be unnecessary, making HUCBCs an ideal candidate for cell-based products. Furthermore, as the technology for banking HUCBCs improves, it is possible that autologous transplantation (i.e., transplantation of an individual's own cells back into that person's body) will be plausible. This would completely eliminate the need for immunosuppression during cellular therapy, which is utilized to prevent rejection but is a very difficult process to management successfully.

Intravenously administered HUCBCs preferentially survive and differentiate into neurons in the damaged brain, and promote behavior recovery in preclinical models of CVA. While intravenous delivery of HUCBCs has promoted functional recovery in preclinical models of CVA, the behavioral improvements are only partial, leaving significant room for increments in the efficacy of these cells in vivo.

Because of the difficulty in effectively treating patients after stroke and other ischemic events, there is a need in the art for methods to enhance the treatment of ischemic and inflammatory events, particularly CVA.

### BRIEF SUMMARY OF THE INVENTION

It is an object of the present invention to optimize the use of human umbilical cord blood cells in patients with inflammation.

In one embodiment there is disclosed a method for repairing animal tissue damage due to an inflammatory reaction in an animal that has the steps of providing umbilical cord blood cells (UCBCs) in a pharmaceutically acceptable form; and administering a sufficient dose of UCBC at an optimal time, thereby reducing the injury from the inflammatory reaction. The optimal time is 48 hours, more than about 24 hours, about 26 hours, about 28 hours, about 30 hours, about 32 hours, about 35 hrs, about 38 hours, about 40 hours, about 42 hours, about 44 and about 46 hours. The optimal time is less than 72 hours, about 70 hours, about 68 hours, about 65 hours, about 62 hours, about 60 hours, about 58 hours, about 55 hours, about 52 hours and about 50 hours.

In another embodiment, the optimal time is between about 26 hours and about 70 hours, between about 28 hours and about 68 hours, between about 30 and 65 hours, between about 32 hours and about 62 hours, between about 32 hours and 60 hours, between about 35 hours and about 58 hours, between about 38 hours and about 55 hours, between about 40 hours and about 52 hours, between about 42 hours and about 50 hours, or between about 45 hours and about 48 hours.

In one embodiment, the UBCBs are administered by a parenteral route. Alternately, the UCBCs are administered intravenously, intra-arterially, intramuscularly, subcutaneously, transdermally, intratracheally, intraperitoneally or into spinal fluid. Alternately, the UCBCs are administered to the site of inflammation or injury, or into an ischemic area, particularly in the brain.

In yet another embodiment, the UCBCs are administered in an amount sufficient to treat the particular site and size of the inflammation or injury. Alternately, the UCBCs are administered in a sufficient amount, factoring in the route of administration.

In still another embodiment, there is a method of treating a patient's Multiple Sclerosis after a flare-up, comprising administering to the patient within 48 hours of a flare-up a sufficient quantity of human umbilical cord blood cells (HUCBCs) into the spinal fluid or bloodstream. Alternately the method further comprises delivering the HUCBCs into the spinal fluid by way of an implanted pump.

In yet another embodiment, there is a method for treating acute central nervous system inflammation in a patient that calls for administering a sufficient quantity of HUCBC in a physiologically compatible solution to an individual suffering from an acute central nervous system inflammation. The condition in which the HUCBCs are administered is meningitis, trauma or cerebrovascular accident (CVA). The CVA can be thrombolic or hemorrhagic.

In yet another embodiment, HUCBCs are administered is in the range of about 10⁵ to about 10¹³ and are administered at about 48 hours. Alternately, the quantity of HUCBCs administered is 5×10⁶ per kilogram and is administered at about 48 hours.

In another embodiment, there is provided a method of treating myocardial ischemia in an individual by providing HUCBCs in a physiological solution; and administering the HUCBCs to the individual experiencing myocardial ischemia at a time that is 2-24 hrs after the onset of ischemia.

In yet another embodiment, there is a method of treating bronchopulmonary distress in a neonate by providing HUCBCs in a physiological solution; and administering the HUCBCs to the individual experiencing myocardial ischemia at a time that is 2-24 hrs after the onset of ischemia.

In still another embodiment there is a kit for determining when HUCBCs should be administered to an individual with an inflammatory condition, the kit having at least one container containing antibodies specific for IL-8 and MCP-1; and directions for obtaining and preparing a tissue sample, directions for performing a test of IL-8 and MCP-1 in the sample, and directions for interpreting the amounts of IL-8 and MCP-1 in the sample. Alternately the kit can have two containers, one containing antibody to IL-8 and one containing antibody to MCP-1. The tested tissue can be blood, spinal fluid, biopsy, or bronchial lavage. Additionally, the kit can include antibodies to TIMP-1 and β-NGF, the former being a control to MCP-1 and IL-8 and the latter indicating a later marker of inflammation.

### BRIEF DESCRIPTION OF DRAWINGS

FIGS. 1A-1C are graphs. FIG. 1A shows the success of the rats in the post-surgical step test compared to pre-surgical baseline. All step test results were markedly decreased except those of the 48 hr, HUCBC-treated group. FIG. 1A is a scatter gram comparing the percent steps at baseline to the percent of intact tissue volume of the ipsilateral stroked side compared to the contralateral side. Generally, the greater is the brain volume (e.g., for 48 hr treatment), the greater is the percent of baseline steps, indicating substantial improvement. In FIG. 1C, the rats receiving a transplant 48 hr after MCAO showed significantly greater motor improvement one month post-transplant than MCAO-only controls.

FIGS. 2A-2G are photomicrographs of rat brain taken at 0.3 mm posterior to the bregma to show the extent of the infarction after middle cerebral artery occlusion (MCAO), an established model for CNS ischemic injury, commonly assessed by microscopic evaluation of striatal and hippocampal tissue. All showed significant CVA damage on the operated, ipsilateral side, except FIG. 2D, which came from a rat treated with HUCBCs 48 hrs after MCAO.

FIG. 3 is a bar graph indicating significantly greater loss of striatal and cortical cells on the ipsilateral, operated side compared to the contralateral, normal side, except at 48 hr transplantation, indicating that HUCBC transplantation at 48 hr reverses the usual course of ischemic destruction of viable tissue.

FIGS. 4A-4J are photomicrographs showing the different cell types in MCAO lesions and adjacent tissue. FIGS. 4A and 4B show tissue stained with antibody against glial fibrillary acidic protein (GFAP) for astrocytes. As expected, more intense staining (bright color) in the MCAO-only control (FIG. 4A, scale bar=50 μm) lesion than in the lesion treated with HUCBCs at 48 hr (FIG. 4B, scale bar=50 μm) was observed. Photomicrographs FIGS. 4C-4D were stained with anti-rat MHC II antibody (O×6). MCAO-only animals exhibited extensive O×6 immunolabeling; whereas, animals treated with HUCBCs at 48 hrs following surgery had no significant staining (FIG. 4D, scale bar=200 μm). For photomicrographs FIGS. 4E and 4F, dead and degenerating neurons were identified with fluorojade (Histochem, Inc., Jefferson, Ariz.); intense staining in FIG. 4E (scale bar=200 μm) indicates cell death in the MCAO only cells, in contrast to the lesser staining in the HUCBC-treated at 48 hr in FIG. 4F (scale bar=200 μm). In photomicrographs FIGS. 4G to 4J, the cellular esterase active of granulocytes was labeled with naphthol AS-D chloroacetate esterase and that of monocytes with α-naphthyl acetate esterase. MCAO-only controls (FIG. 4G, scale bar=100 μm) exhibited more intense staining of monocytes than 48 hr HUCBC-treated animals (FIG.4H, scale bar=100 μm). More granulocytes were found in the MCAO only controls (FIG. 4I, scale bar=500 μm) than 48 hr-treated rats (FIG. 4J, scale bar=50 μm). In contrast, granulocytes found in the 48hr treated rats were largely confined to vessels.

FIGS. 5A-5F are photomicrographs of sections prepared to show apoptosis. FIG. 5A is obtained from a sham-operated negative control. FIG. 5B shows cells undergoing apoptosis in the core at their peak at 2 days after MCAO; many apoptotic cells were still seen at day 4 (FIG. 5C) and day 7 (FIG. 5D). Importantly, when HUCBCs were given at 48 hr, no apoptotic cells were observed at days 4 and 7 (FIGS. 5E and 5F). Scale bar=100 μm.

FIGS. 6A-6F, 6A¹-6F¹, 6A²-6F² are photomicrographs; and FIG. 6G is a graph depicting the total, non-infarct volume of the ischemic, ipsilateral hemisphere compared to the total volume of the contralateral hemisphere. FIGS. 6A-6D illustrate that the infarct progression appears to continue over the course of 7 days following MCAO: 6A is sham-operated, 6B is MCAO operation only at 2 days, 6C is MCAO only at 4 d, 6D is MCAO only at 7 d. Clearly rats sacrificed at 2 d after MCAO showed minimal pathologic damage, supporting the conclusion that neurons remain potentially viable at this point. When the 4- and 7-day results for HUCBC-transplanted rats (FIGS. 6E and 6F, respectively) are compared, infarct progression was significantly arrested (p=0.014). FIG. 6G illustrates comparative sizes of the ipsilateral and contralateral hemispheres. MCAO-only rats sacrificed at 2, 4 and 7 d had significantly greater infarct volumes than sham-operated which were analyzed by the Mann Whitney test (U=0, p=0.0039; U=0, p=0.0027; U=0, p=0.0039, respectively). MCAO-only rats sacrificed at 4 and 7 d also had significantly greater infarct volumes than HUCBC-treated rats sacrificed 7 days post-CVA (U=6, p=0.0181; U=5, p=0.0223, respectively). Moreover, immunohistochemistry of astrocytes and activated microglia showed that both inflammatory cell types increased in number up to 4 d post-MCAO (6B1, 6C1 and 6B2, 6C2, respectively), diminishing by 7 d (6D1 and 6D2, respectively). Scale bar=100 μm.

FIG. 7 is a bar graph showing the human cytokines produced by HUCBCs by increasing concentrations of HUCBCs. The graph represents the effect of seeding density on cytokine production in cultured HUCBCs. Based on the array membrane technique, only the five shown cytokines differed from the negative control (DMEM). Cytokines showed a progressive increase in optical density that corresponded to the concentration of HUCBCs plated.

FIGS. 8A-8D are radiographs of human cytokine arrays. HUCBCs were cultured for four days in Ex Vivo 10 solution (Cambrex, Walkersville, Md.) with no serum, then supplemented with IL-3, thrombopoietin (TPO) or nothing for 5 days, after which media were changed to plain Ex Vivo 10 solution for an additional three days. FIG. 8A is the negative control (Ex Vivo 10 medium alone); FIG. 8B represents conditioned medium from HUCBCs treated with IL-3 (5 ng/mL); FIG. 8C represents conditioned medium from HUCBCs treated with TPO (25 ng/mL); and FIG. 8D represents conditioned medium from 50 million HUCBCs in Ex Vivo 10 solution without other biologics. Several cytokines were present in conditioned media of HUCBCs under the various culturing conditions. Most importantly, a different medium (Ex Vivo 10) from DMEM induced the release of several different cytokines. Ex Vivo 10 is a serum-free medium originally designed to support hematopoietic cells in long-term culture. IL-8 was strongly released in all conditions except the control. IL-8 increases in ischemic CVA but has not been previously reported in conditioned medium from HUCBCs. IL-8 attracts neutrophils. Overall, this assay shows that HUCBCs can be induced to release cytokines.

FIG. 9 is a listing of cytokine names shown in FIGS. 8A-8B, in the order of intensity on each radiograph. OSM is oncostatin M, PDGFb is platelet derived growth factor, RANTES is regulated upon activation normal T-cell expressed and secreted, TNF is tumor necrosis factor, MIG is monokine induced by interferon γ. MDC is macrophage derived chemokine, and GRO is growth regulated oncogene.

FIGS. 10A and 10B are radiographs showing cytokines in conditioned medium from HUCBCs (10A) and plain medium controls (10B). The HUCBCs released IL-8, MCP-1, ENA78 and MDC.

FIGS. 11A-11D are radiographs of cytokines in rat striatal tissue from MCAO-treated rats. FIG. 11A shows the 12-hr contralateral side (unoperated); FIG. 11B shows the 12-hr operated side; FIG. 10C shows the 48 hr contralateral; and FIG. 11D shows the 48-hr ipsilateral side. TIMP-1 was released from all samples. The 48-hr ipsilateral side attracted HUCBCs and responded favorably to their administration by releasing MCP-1 and GRO/CINC-1. This suggests that these proteins may be important in the beneficial effect of HUCBCs.

FIGS. 12A-12D show additional cytokine radiographs of rat striatal tissue from MCAO-treated rats. FIG. 12B (1-wk ipsilateral) still shows MCP-1 and GRO/CINC-1, but also has β-NGF, however at a less intense level.

FIGS. 13A and 13B are graphs showing the time course of GRO/CINC-1 in MCAO rat striatal and hippocampal (respectively) extracts from rats sacrificed at 4, 6, 12, 24, 48 and 72 hr and 1 wk after surgery. The increases in the chemokines MCP-1 and GRO/CINC-1 (the rat version of IL-8), which will enhance migration to the site of the brain lesion, occur between 12 to 72 hours. This time course brackets the time course of behavioral and anatomical improvements observed with HUCBC transplants at 48 hours.

FIGS. 14A and 14B are graphs showing the time course of MCP-1 in MCAO rat striatal and hippocampal (respectively) extracts from rats sacrificed at 4, 6, 12, 24, 48 and 72 hours and 1 week after surgery.

FIG. 15 is a series of bar graphs showing the luminescence expression of labeled ATP for treatments of HUCBCs with various concentrations of MCP-1, IL-3 and TPO.

FIGS. 16A-16H are bright field photomicrographs of HUCBCs that have migrated to MCAO tissue extracts and controls. Pictures were taken of the bottom well in the 96-well plate on an inverted microscope at 10×. FIGS. 16A, 16B and 16E show the numerous HUCBCs that migrated to striatal and hippocampal extracts at both 24 hr and 72 hr after MCAO. Upon reaching the bottom plate, the cells began to form cell clusters in these conditions. FIGS. 16B, 16D, 16F and 16H show that very few cells migrated to the control conditions and this was typical for all samples tested. FIG. 16G shows that HUCBCs also migrated to chemoattractant SDF-1; however, the pattern of migration was more random with no defined cell clusters. For all photomicrographs, bar=100.

FIGS. 17A-17D are bar graphs showing the relative numbers of HUCBCs that migrated to MCAO brain tissue extracts at 4, 6, 12, 24, 48 and 72 hr and 1 wk after ischemia and to controls. FIG. 17A shows that a significant number of HUCBCs that migrated to the stroked striatal tissue extracts at 24 hr (*) and to the stroked striatal and FIG. 17B shows the hippocampal tissue extracts at 48 and 72 hr conditions (*) when compared to tissue extracts from non-stroke side or plain media. Interestingly at 4, 6 and 12 hrs, significantly fewer cells (#) migrated to the strial stroke tissue extracts compared to non-stroked control. At 4 and 6 hr after MCAO, fewer cells (#) migrated to hippocampal stroke tissue extracts compared to non-stroke extracts, while at 24 hr, fewer cells migrated to the stroke side when compared to non-stroke side. Significantly more HUCBCs (**) migrated to SDF-1 chemoattractant and few cells migrated to the medium control (#) compared to all other conditions.

The following written description provides exemplary methodology and guidance for carrying out many of the varying aspects of the present invention.

### DETAILED DISCLOSURE OF THE INVENTION

Every 45 seconds someone in the United States experiences a CVA, and every 3 minutes someone dies from one. Currently, the thrombolytic tissue plasminogen activator (TPA) is the only FDA-approved treatment CVA. However, TPA has considerable limitations in that it is only effective if used within 3 hours of CVA onset, can only be used with embolic CVA, and, even more, can have devastating side effects. A safer, more inclusive treatment is needed which opens the temporal window for therapeutic possibility to ensure that our growing elderly population survive and suffer fewer deficits after CVA.

Transplantation of the mononuclear fraction of human umbilical cord blood cells (HUCBCs) have been used, successfully, to treat spinal cord injury (Saporta et al., 2003 J Hematother Stem Cells Res, June,12(3):271-8), traumatic brain injury (Lu et al., 2002, Cell Transplant 11(3):275-81) and neurodegenerative diseases and injury (Garbuzova-Davis et al., 2003, J Hematother Stem Cells Res, 12(3): 255-70; Newman et al., 2003a, Neurotox Res 5(5):355-68; Willing et al., 2003a, J Neurosci Res 8/1 73(3):296-307; Willing et al., 2003b, Cell Transplant 12(4):449-54) Recent studies of intravenous administration of HUCBCs in a rat model of Middle Cerebral Artery Occlusion (MCAO) have demonstrated improved performance in tests of motor coordination when HUCB was given 24 hr after MCAO (Chen et al., 2001, Stroke 32(11):2682-8; Willing et al., 2003a,b, ibid.). Though this treatment time point is sufficient for noticeable recovery there is in vitro evidence that delivery of cells at 48 and 72 hrs may be more effective (Newman et al., 2003b, ibid.). It is at this time that the astrocytic and microglial inflammatory response to ischemia are at their peak as well as signals that attract HUCBCs to migrate to the site of injury. In the present study the optimal time at which intravenous administration of HUCBCs was most beneficial to behavioral recovery and neurophysiologic repair was proven.

Unless otherwise noted, the terms used herein are to be understood according to conventional usage by those of ordinary skill in the relevant art.

In addition to the definitions of terms provided below, definitions of common terms in molecular biology may also be found in Rieger et al., 1991 GLOSSARY OF GENETICS: CLASSICAL AND MOLECULAR, 5th Ed., Berlin: Springer-Verlag; and in CURRENT PROTOCOLS IN MOLECULAR BIOLOGY, F. M. Ausubel et al., Eds., Current Protocols, a joint venture between Greene Publishing Associates, Inc. and John Wiley & Sons, Inc. (1998 Supplement).

It is to be understood that as used in the specification and in the claims, “a” or “an” can mean one or more, depending upon the context in which it is used. Thus, for example, reference to “a cell” means at least one cell.

The present invention may be understood more readily by reference to the following detailed description of the preferred embodiments of the invention and the Examples included herein. However, before the present compounds, compositions, and methods are disclosed and described, it is to be understood that this invention is not limited to specific nucleic acids, specific polypeptides, specific cell types, specific host cells, specific conditions, or specific methods, etc., as such may, of course, vary, and the numerous modifications and variations therein will be apparent to those skilled in the art. It is also to be understood that the terminology used herein is for the purpose of describing specific embodiments only and is not intended to be limiting.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al., 1989 MOLECULAR CLONING, Second Edition, Cold Spring Harbor Laboratory, Plainview, N.Y.; Maniatis et al., 1982 MOLECULAR CLONING, Cold Spring Harbor Laboratory, Plainview, N.Y.; Wu (Ed.) 1993 Meth. Enzymol. 218, Part I; Wu (Ed.) 1979 Meth Enzymol. 68; Wu et al., (Eds.) 1983 Meth. Enzymol. 100 and 101; Grossman and Moldave (Eds.) 1980 Meth. Enzymol. 65; Miller (ed.) 1972 EXPERIMENTS IN MOLECULAR GENETICS, Cold Spring Harbor Laboratory, Cold Spring Harbor, N.Y.; Old and Primrose, 1994 PRINCIPLES OF GENE MANIPULATION, 5^{th} ed., University of California Press, Berkeley; Schleif and Wensink, 1982 Practical Methods in Molecular Biology; Glover (Ed.) 1985 DNA CLONING: VOLS. I AND II, IRL Press, Oxford, UK; Hames and Higgins (Eds.) 1985 NUCLEIC ACID HYBRIDIZATION, IRL Press, Oxford, UK; and Setlow and Hollaender 1979 GENETIC ENGINEERING: PRINCIPLES AND METHODS, Vols. 1-4, Plenum Press, New York. Abbreviations and nomenclature, where employed, are deemed standard in the field and commonly used in professional journals such as those cited herein. For the convenience of the reader, a list follows:
BDNF, brain derived neurotrophic factor
BMP, bone-morphogenetic proteins
CNTF, ciliary neurotrophic factor
CSF, cerebral spinal fluid, colony stimulating factors
CVA, cerebrovascular accident
DMEM, Dulbecco's modified Eagle medium
ENA-78, epithelial cell-derived neutrophil activating protein
FBS, fetal bone serum
FGF, fibroblast growth factor
GDNF, glial derived neurotrophic factor
GGF, glial growth factor
GRO, growth regulated oncogene
GRO/CINC-1, growth-related oncogene/cytokine-induced neutrophil chemoattractant
HRP, Streptavidin horseradish peroxidase
HUCBC, human umbilical cord blood cell
IGF, insulin-like growth factor
LIF, leukemia inhibitory factory
MCAO, middle cerebral artery occlusion
MCP-1, monocytes-chemoattractant protein 1
MDC, macrophage derived chemokine
MIG, monokine induced by interferon y
NGF, nerve growth factor
OFNE, oxygenated fluorocarbon nutrient emulsion
OSM, oncostatin M
PBS, phosphate buffered saline
PDGFb, platelet derived growth factor
RANTES, regulated upon activation normal T-cell expressed and secreted
RDS, respiratory distress syndrome
RMP-7, receptor-mediated permeabilizer
TdT, terminal deoxynucleotidyl transferase
TGF, transforming growth factor
TNF, tumor necrosis factor
TPA, tissue plasminogen activator
TPO, thrombopoietin
TUNEL, TdT deoxyuridine nicked end labeling
UCB, umbilical cord blood
UCBC, umbilical cord blood cells

The HUCBC of the subject invention can be administered to patients, including veterinary (e.g., mammalian) patients, to alleviate the symptoms of a variety of pathological conditions for which cell therapy is applicable. For example, the cells of the present invention can be administered to a patient to alleviate the symptoms of acute, subacute and chronic neurological disorders such as CVA (e.g., transient ischemic attacks [TIA], hypoxia-ischemia); neurodegenerative diseases, such as Huntington's disease, Alzheimer's disease, and Parkinson's disease; traumatic brain injury; spinal cord injury; epilepsy (e.g., seizures and convulsions); Tay Sach's disease (β-hexosaminidase A deficiency); lysosomal storage disease; amyotrophic lateral sclerosis; meningitis; multiple sclerosis (MS) and other demyelinating diseases; neuropathic pain; Tourette's syndrome; ataxia, drug addiction, such as alcoholism; drug tolerance; drug dependency; depression; anxiety; and schizophrenia. In a preferred embodiment of the present invention, the cells are administered to alleviate the symptoms of CVAs.

The present invention is also directed to a method of treating neurological damage in the brain or spinal cord which occurs as a consequence of genetic defect, physical injury, environmental insult or damage from a CVA, heart attack or cardiovascular disease in patients, the method comprising administering (including transplanting), an effective number, volume or amount of HUCBCs to patients at a time point specifically determined to provide optimal therapeutic efficacy.

In one embodiment, the administration of umbilical cord blood cells at a time point specifically determined to provide therapeutic efficacy leads to a determination that 2-3 days after an ischemic event monocyte chemoattractant protein-1 (MCP-1) expression is at its peak having been stimulated by IL-1, TNFα, IFNγ, LPS and platelet derived growth factor. MCP-1 is highly specific to monocytes and is expressed by endothelial cells and macrophages (Chen et al., 2001, ibid.; Yamagami et al., 1999, J Leukoc Biol 65:744-9). Prior to 48 hrs MCP-1 signals may not be strong enough to attract HUCBC and after 48 hrs it may be too late for cells in the ischemic core to recover.

The pharmaceutical compositions may further comprise a neural cell differentiation agent. The pharmaceutical compositions may further comprise a pharmaceutically acceptable carrier.

The term “patient” is used herein to describe an animal, preferably a human, to whom treatment, including prophylactic treatment, with the cells according to the present invention, is provided. For treatment of those conditions or disease states which are specific for a specific mammal such as a human patient, the term patient refers to that specific mammal.

The term “donor” is used to describe an individual (particularly a mammalian animal, including a human) who donates umbilical cord blood or umbilical cord blood cells for use in a recipient or patient.

The term “umbilical cord blood” (UCB) is used herein to refer to blood obtained from the umbilical cord and/or placenta, most preferably from a neonate. Preferably, the umbilical cord blood is isolated from human newborn umbilical cord and/or placenta. The use of umbilical cord blood as a source of mononuclear cells is advantageous because it can be obtained relatively easily and without trauma to the donor. In contrast, the collection of bone marrow cells from a donor is a traumatic experience. Umbilical cord blood cells (UCBCs) can be used for autologous transplantation or allogeneic transplantation, when and if needed. Umbilical cord blood is preferably obtained by direct drainage from the cord and/or by needle aspiration from the delivered placenta at the root and at distended veins.

As used herein, the term “human umbilical cord blood cells” (HUCBCs) refers to cells that are present within human umbilical cord blood and placenta. In one embodiment, the HUCBCs include a fraction of the UCB, containing mainly mononuclear cells that have been isolated from the umbilical cord blood using methods known to those skilled in the art. In a further embodiment, the HUCBCs may be differentiated prior to administration to a patient.

The term “effective amount” is used herein to describe concentrations or amounts of components such as differentiation agents, umbilical cord blood cells, precursor or progenitor cells, specialized cells, such as neural and/or neuronal or glial cells, blood brain barrier permeabilizers and/or other agents which are effective for producing an intended result including differentiating stem and/or progenitor cells into specialized cells, such as neural, neuronal and/or glial cells, or treating a neurological disorder or other pathologic condition including damage to the central nervous system of a patient, such as a CVA, heart attack, or accident victim or for effecting a transplantation of those cells within the patient to be treated. An effective amount can be determined for hypoxic neonates requiring high-dose oxygen therapy. Compositions according to the present invention may be used to effect a transplantation of the umbilical cord blood cells within the composition to produce a favorable change in the brain or spinal cord, or in the disease or condition being treated, whether that change is stabilization, an improvement (such as stopping or reversing the degeneration of a disease or condition being treated, such as reducing a neurological deficit or improving a neurological response) or a complete cure of the disease or condition treated.

The terms “stem cell” or “progenitor cell” are used interchangeably herein to refer to umbilical cord blood-derived stem and progenitor cells. The terms stem cell and progenitor cell are known in the art (e.g., STEM CELLS: SCIENTIFIC PROGRESS AND FUTURE RESEARCH DIRECTIONS, report from the National Institutes of Health, June, 2001). The term “neural cells” are cells having at least an indication of neuronal or glial phenotype, such as staining for one or more neuronal or glial markers or which will differentiate into cells exhibiting neuronal or glial markers. Examples of neuronal markers which may be used to identify neuronal cells according to the present invention include, for example, neuron-specific nuclear protein, tyrosine hydroxylase, microtubule associated protein, and calbindin, among others. The term neural cells also includes cells which are neural precursor cells, i.e., stem and/or progenitor cells which will differentiate into or become neural cells or cells which will ultimately exhibit neuronal or glial markers, such term including pluripotent stem and/or progenitor cells which ultimately differentiate into neuronal and/or glial cells. All of the above cells and their progeny are construed as neural cells for the purpose of the present invention. Neural stem cells are cells with the ability to proliferate, exhibit self-maintenance or renewal over the lifetime of the organism and to generate clonally related neural progeny. Neural stem cells give rise to neurons, astrocytes and oligodendrocytes during development and can replace a number of neural cells in the recipient brain. Neural stem cells are neural cells for purposes of the present invention. The terms “neural cells” and “neuronal cells” are generally used interchangeably in many aspects of the present invention. Preferred neural cells for use in certain aspects according to the present invention include those cells which exhibit one or more of the neural/neuronal phenotypic markers such as Musashi-1, Nestin, NeuN, class III β-tubulin, GFAP, NF-L, NF-M, microtubule associated protein (MAP2), S100, CNPase, glypican (especially glypican 4), neuronal pentraxin II, neuronal PAS 1, neuronal growth associated protein 43, neurite outgrowth extension protein, vimentin, Hu, internexin, Oct₄, myelin basic protein and pleiotrophin, among others.

The term “administration” or “administering” is used throughout the specification to describe the process by which cells of the subject invention, such as umbilical cord blood cells obtained from umbilical cord blood, or differentiated cells obtained therefrom, are delivered to a patient for therapeutic purposes. Cells of the subject invention are administered a number of ways including, but not limited to, parenteral, intrathecal, intraventricular, intraparenchymal (including into the spinal cord, brainstem or motor cortex), intracisternal, intracranial, intrastriatal, and intranigral, among others. Basically any method can be used so that it allows cells of the subject invention to reach the ultimate target site. Cells of the subject invention can be administered in the form of intact umbilical cord blood or a fraction thereof (such term including a mononuclear fraction thereof or a fraction of mononuclear cells, including a high concentration of stem cells). The compositions according to the present invention may be used without treatment with a mobilization agent or differentiating agent (“untreated” i.e., without further treatment in order to promote differentiation of cells within the umbilical cord blood sample) or after treatment (“treated”) with a differentiation agent or other agent which causes certain stem and/or progenitor cells within the umbilical cord blood sample to differentiate into cells exhibiting a differentiated phenotype, such as a neuronal and/or glial phenotype. The cells may undergo ex vivo differentiation prior to administration into a patient.

The umbilical cord blood stem cells can be administered systemically or to a target anatomical site, permitting the cells to differentiate in response to the physiological signals encountered by the cell (e.g., site-specific differentiation).

Administration often depends upon the disease or condition treated and may preferably be via a parenteral route, for example, intravenously, by administration into the cerebral spinal fluid or by direct implantation into the affected tissue in the brain. For example, in the case of Alzheimer's disease, Huntington's disease, and Parkinson's disease, the preferred route of administration will be a transplant directly into the striatum (caudate putamen) or directly into the substantia nigra (Parkinson's disease). In the case of amyotrophic lateral sclerosis (Lou Gehrig's disease) and multiple sclerosis, the preferred route of administration is injection into the cerebrospinal fluid. In the case of lysosomal storage disease, the preferred route of administration is via an intravenous route or via the cerebrospinal fluid. In the case of CVA, the preferred route of administration will depend upon where the CVA is, but may be directly into the affected tissue (which may be readily determined using MRI or other imaging techniques), or may be administered systemically. In the case of neonatal or older hypoxia, the preferred method of administration also is intravenous, although intratracheal or nasal routes also may be used. In a preferred embodiment of the present invention, the route of administration for treating an individual post-CVA is systemic, via intravenous or intra-arterial administration.

The terms “grafting” and “transplanting” and “graft” and “transplantation” are used throughout the specification synonymously to describe the process by which cells of the subject invention are delivered to the site where the cells are intended to exhibit a favorable effect, such as repairing damage to a patient's central nervous system (which can reduce a cognitive or behavioral deficit caused by the damage), treating an acute or subacute neurodegenerative disease, nerve damage caused by CVA, physical injury, trauma, or environmental insult to the brain and/or spinal cord, caused by, for example, an accident or other activity. Cells of the subject invention can also be delivered in a remote area of the body by any mode of administration as described above, relying on cellular migration to the appropriate area to effect transplantation. In one embodiment, the cells are co-administered with a blood brain barrier permeabilizer, such as mannitol or RMP-7 receptor-mediated permeabilizer that is a peptide bradykinin analog.

The term “non-tumorigenic” refers to the fact that the cells do not give rise to a neoplasm or tumor. Stem and/or progenitor cells for use in the present invention are most preferably free from neoplastic and cancerous cells.

The term “differentiating agent” or “neural differentiating agent” is used throughout the specification to describe agents which may be added to cell culture (which term includes any cell culture medium which may be used to grow neural cells according to the present invention) containing umbilical cord blood pluripotent or multipotent stem and/or progenitor cells which will induce the cells to a more differentiated phenotype, such as a neuronal or glial phenotype. Preferred differentiation agents for use in the present invention include, for example, antioxidants, including retinoic acid, fetal or mature neuronal cells including mesencephalic or striatal cells or a growth factor or cytokine such as brain derived neurotrophic factor (BDNF), glial growth factor (GFF), glial derived neurotrophic factor (GDNF) and nerve growth factor (NGF) or mixtures, thereof. Additional differentiation agents include, for example, growth factors such as fibroblast growth factor (FGF), transforming growth factors (TGF), ciliary neurotrophic factor (CNTF), bone-morphogenetic proteins (BMP), leukemia inhibitory factor (LIF), glial growth factor (GGF), tumor necrosis factors (TNF), interferon, insulin-like growth factors (IGF), colony stimulating factors (CSF), KIT receptor stem cell factor (KIT-SCF), interferon, triiodothyronine, thyroxine, erythropoietin, thrombopoietin, silencers, (including glial-cell missing, neuron restrictive silencer factor), SHC (SRC-homology-2-domain-containing transforming protein), neuroproteins, proteoglycans, glycoproteins, neural adhesion molecules, and other cell-signaling molecules and mixtures, thereof Differentiating agents which can be used in the present invention are detailed in “Marrow-mindedness: a perspective on neuropoiesis” by Bjorn Scheffler et al., TINS, 1999, 22:348-356, which is incorporated by reference herein in its entirety.

The term “neurodegenerative disease” is used herein to describe a disease which is caused by damage to the central nervous system and which damage can be reduced and/or alleviated through transplantation of neural cells according to the present invention to damaged areas of the brain and/or spinal cord of the patient. Exemplary neurodegenerative diseases which may be treated using the neural cells and methods according to the present invention include for example, Parkinson's disease, Huntington's disease, amyotrophic lateral sclerosis (Lou Gehrig's disease), Alzheimer's disease, Rett Syndrome, lysosomal storage disease (“white matter disease” or glial/demyelination disease, as described, for example by Folkerth, 1999, J Neuropath Exp Neuro, September, 58:9), including Sanfilippo, Gaucher disease, Tay Sachs disease (β-hexosaminidase A deficiency), other genetic diseases and disorders, multiple sclerosis flare-ups, brain injury or trauma caused by ischemia, accidents, environmental insult, etc., spinal cord damage and drug dependency such as alcoholism. In addition, the present invention may be used to reduce and/or eliminate the effects on the central nervous system of a CVA or a heart attack in a patient, which is otherwise caused by lack of blood flow or ischemia to a site in the brain of said patient or which has occurred from physical injury to the brain and/or spinal cord. Neurodegenerative diseases also include neurodevelopmental disorders including for example, Tay-Sachs disease.

The subject cells also are used in other types of inflammation, preferably at such a time that cells native to the inflamed area have not been killed by the inflammatory process. Examples include but are not limited to neonatal bronchopulmonary dysplasia (BPD), respiratory distress syndrome (RDS) and myocardial infarction, ischemia and angina.

The term BPD refers to a type of inflammatory over-reaction that may develop in utero or be diagnosed shortly after birth. Neonates at highest risk are those with low birth weights (especially less than about 1.5 kg) and who are premature (especially less than 30 weeks gestation). When BPD is diagnosed, neonates must remain in the hospital for months or longer, resulting in susceptibility to infection, poor growth and huge medical bills, as ventilated neonates must stay in the neonatal ICU. Artificial ventilation with high oxygen values (e.g., 1.0 vs 0.2, which is room air) can exacerbate the condition through hyperoxia or oxygen toxicity. In preterm infants, BPD may start as early inflammation (due to hyperoxia, infection, etc.), but it is followed by interstitial fibrosis and abnormal bronchopulmonary structure with suppressed development of alveoli, the site of oxygen and carbon dioxide exchange. In preterm infants with respiratory distress syndrome (RDS), during the initial postnatal days, an inflammatory reaction takes place in the lungs characterized by accumulation and activation of inflammatory cells and release of inflammatory mediators in the airways and interstitium. Production of various surfactants may well be affected; the quantity and quality of these surfactants may be compromised. Excessive reparative processes lead to pulmonary fibroproliferation, poor respiration and abnormal lung development. By administering HUCBCs at the time of production of HUCBC attractants (MCP-1 and IL-8), development of fibrosis is minimized. Similar pathology develops in pediatric and adult patients with asthma or hyperreactive pulmonary airway disease, in whom HUCBC treatment can be administered prior to allergy season to reduce excess inflammation, if the bronchoconstriction is caused by exogenous factors which are predictably seasonal in nature. If the asthma or hypereactive bronchopulmonary disease is intrinsic, chronic or periodic, administration of HUCBCs is preferred. If the patient with asthma or hyperreactive bronchopulmonary disease is sensitive to any of a variety of exogenous stimulants, chronic or periodic administration of HUCBCs is appropriate for treatment or prophylaxis.

Acute myocardial infarction (AMI), Prinzmetal's angina pectoris and myocardial ischemia are caused by chronic and/or abrupt occlusion of major coronary arteries, usually caused by rupture of an existing atherosclerotic plaque. All may benefit from standard medical and surgical treatments and administration of HUCBCs to minimize inflammation and repair hypoxic/necrotic myocardial muscle tissue. An AMI generally occurs with the acute rupture of an atherosclerotic plaque causing activation of the blood clotting cascade leading to arterial occlusion, localized hypoxemia or anoxia and subsequent cell damage and/or death. In many instances, the localized area of infarction is extended peripherally through continued hypoxia and inflammatory processes. HUCBCs help repopulate necrotic myocardial muscle cells (i.e., dead cells) and to retard or reverse peripheral extension of the AMI. Prinzmetal's angina pectoris and myocardial ischemia are “chronic” myocardial ischemic conditions caused by slow occlusion, rather than acute occlusion of a cardiac artery. The ischemia associated with both draws administered HUCBCs to the affected site and help the patient by modifying the inflammatory responses and repopulating dysfunction cardiac cells. In vivo research has shown that administering HUCBCs in the time interval between 2 hours and 24 hours was optimal to obtain the maximal beneficial effect (least deterioration of heart function).

The term “gene therapy” is used throughout the specification to describe the transfer and stable insertion of new genetic information into cells for the therapeutic treatment of diseases or disorders. The foreign gene is transferred into a cell that proliferates to spread the new gene throughout the cell population. Thus, umbilical cord blood cells, or progenitor cells are the targets of gene transfer either prior to differentiation or after differentiation to a neural cell phenotype. The umbilical cord blood stem or progenitor cells of the present invention can be genetically modified with a heterologous nucleotide sequence and an operably linked promoter that drives expression of the heterologous nucleotide sequence. The nucleotide sequence can encode various proteins or peptides. The gene products produced by the genetically modified cells can be harvested in vitro or the cells can be used as vehicles for in vivo delivery of the gene products (i.e., gene therapy).

Molecular Biology Techniques

Standard molecular biology techniques known in the art and not specifically described are generally followed as in Sambrook et al., MOLECULAR CLONING: A LABORATORY MANUAL, Cold Springs Harbor Laboratory, NY (1989, 1992), and in Ausubel et al., CURRENT PROTOCOLS IN MOLECULAR BIOLOGY, John Wiley and Sons, Baltimore, Md. (1989). Polymerase chain reaction (PCR) methodology is generally employed as specified as in Jam et al., PCR PROTOCOLS: A GUIDE TO METHODS AND APPLICATIONS, Academic Press, San Diego, Calif. (1999). Reactions and manipulations involving other nucleic acid techniques, unless stated otherwise, are performed as generally described in Sambrook et al., MOLECULAR CLONING: A LABORATORY MANUAL, Cold Springs Harbor Laboratory Press, and methodology as set forth in U.S. Pat. Nos. 4,666,828; 4,683,202; 4,801,531; 5,192,659; and 5,272,057, and incorporated herein by reference. In situ PCR in combination with flow cytometry can be used for detection of cells containing specific DNA and mRNA sequences (e.g., Testoni et al., 1996, Blood, 87:3822).

Standard methods in immunology known in the art and not specifically described herein are generally followed as set forth in Stites et al. (Eds.), BASIC AND CLINICAL IMMUNOLOGY, 8^{th} Ed., Appleton & Lange, Norwalk, Conn. (1994); and Mishell and Shigi (Eds.), SELECTED METHODS IN CELLULAR IMMUNOLOGY, W.H. Freeman and Co., New York (1980).

Immunoassays

In general, immunoassays are employed to assess a specimen for cell surface markers or the like. Immunocytochemical assays are well known to those skilled in the art. Both polyclonal and monoclonal antibodies can be used in the assays. Where appropriate other immunoassays, such as enzyme-linked immunosorbent assays (ELISAs) and radioimmunoassays (RIA), are well known to those skilled in the art and can be used. Available immunoassays are extensively described in the patent and scientific literature. See, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771; and 5,281,521 as well as Sambrook et al., MOLECULAR CLONING: A LABORATORY MANUAL, Cold Springs Harbor, N.Y., 1989. Numerous other references also may be relied on for these teachings.

Antibody Production

Antibodies have attained wide use in the laboratory (as indicated in the following examples) and in clinical medicine. Conveniently, antibodies may be prepared against the immunogen or immunogenic portion thereof (for example, a synthetic peptide based on the sequence) or prepared recombinantly by cloning techniques or the natural gene product and/or portions thereof may be isolated and used as the immunogen. Immunogens can be used to produce antibodies by standard antibody production technology well known to those skilled in the art as described generally in Harlow and Lane, ANTIBODIES: A LABORATORY MANUAL, Cold Spring Harbor Laboratory, Cold Springs Harbor, N.Y. (1988) and Borrebaeck, ANTIBODY ENGINEERING—A PRACTICAL GUIDE by W.H. Freeman and Co., New York City (1992). Antibody fragments may also be prepared from the antibodies and include Fab and F(ab′)2 by methods known to those skilled in the art. To produce polyclonal antibodies a host, such as a rabbit or goat, is immunized with the immunogen or immunogenic fragment, generally with an adjuvant and, if necessary, coupled to an immunogenic carrier. Subsequently, antibodies specific to the immunogen are collected from the serum. Furthermore, the polyclonal antibody can be adsorbed such that it is monospecific. That is, the serum can be exposed to related immunogens so that cross-reactive antibodies are removed from the serum rendering it monospecific (i.e., the serum can be exposed to related immunogens so that cross-reactive antibodies are removed from the serum rendering the harvested antibodies).

To produce monoclonal antibodies, an appropriate donor (usually mammalian) is hyperimmunized with the immunogen, and splenic antibody-producing cells are isolated. These cells are fused to immortal cells, such as myeloma cells, to provide a fused hybrid cell line that is immortal and secretes the desired antibody. The cells are then cultured, and the monoclonal antibodies are harvested from the culture medium.

For producing recombinant antibodies, messenger RNA from antibody-producing B-lymphocytes of animals or hybridomas is reverse-transcribed to obtain complementary DNAs (cDNAs). Antibody cDNA, which encodes full or partial length antibody, is amplified and cloned into a phage or a plasmid. The cDNA can encode for be a partial length of heavy and light chain cDNA, separated or connected by a linker. The antibody, or antibody fragment, is expressed using a suitable expression system. Antibody cDNA can also be obtained by screening pertinent expression libraries. The antibody can be bound to a solid support substrate or conjugated with a detectable moiety or be both bound and conjugated as is well known in the art. (For a general discussion of conjugation of fluorescent or enzymatic moieties, see Johnstone & Thorpe, IMMUNOCHEMISTRY IN PRACTICE, 3d ed., Blackwell Scientific Publications, Oxford, 1996). The binding of antibodies to a solid support substrate is also well known in the art. (see for a general discussion Harlow & Lane, ANTIBODIES: A LABORATORY MANUAL, Cold Spring Harbor Laboratory Publications, New York, 1988; and Borrebaeck, ANTIBODY ENGINEERING—A PRACTICAL GUIDE, W.H. Freeman and Co., 1992). The detectable moieties contemplated with the present invention can include, but are not limited to, fluorescent, metallic, enzymatic and radioactive markers. Examples include biotin, gold, ferritin, alkaline phosphates, galactosidase, peroxidase, urease, fluorescein, rhodamine, tritium, ¹⁴C, iodination and green fluorescent protein.

Gene Therapy

Gene therapy as used herein refers to the transfer of genetic material (e.g., DNA or RNA) of interest into a host to treat or prevent a genetic or acquired disease or condition. The genetic material of interest encodes a product (e.g., a protein, polypeptide, peptide, functional RNA, and/or antisense molecule) whose in vivo production is desired. For example, the genetic material of interest encodes a hormone, receptor, enzyme polypeptide or peptide of therapeutic value. Alternatively, the genetic material of interest encodes a suicide gene. For a review see “Gene Therapy” in ADVANCES IN PHARMACOLOGY, Academic Press, San Diego, Calif., 1997.

Administration of Cells for Transplantation

The umbilical cord blood cells of the present invention can be administered and dosed in accordance with good medical practice, taking into account the clinical condition of the individual patient, the site and method of administration, scheduling of administration, patient age, sex, body weight and other factors known to medical practitioners. The pharmaceutically “effective amount” or dosage schedule for purposes herein is to be determined by such considerations as are known to those skilled in the experimental research, pharmacological and clinical medical arts. The amount must be effective to achieve stabilization, improvement (including but not limited to improved survival rate or more rapid recovery) or improvement or elimination of symptoms and other indicators as are selected as appropriate measures by those skilled in the art.

In the method of the present invention, the HUCBCs of the present invention can be administered in various ways as would be appropriate to implant in the central nervous system, including, but not limited to, parenteral administration, including intravenous and intraarterial administration, intrathecal administration, intraventricular administration, intraparenchymal, intracranial, intracistemal, intrastriatal, and intranigral administration.

Optionally, the HUCBCs are administered in conjunction with an immunosuppressive agent, such as cyclosporine, or a BBB permeabilizer, such as mannitol or RMP-7.

Pharmaceutical compositions comprising effective amounts of umbilical cord blood cells are also contemplated by the present invention. These compositions comprise an effective number of cells, optionally, in combination with a pharmaceutically acceptable carrier, additive or excipient and suspended in one or more appropriate liquid media. In certain aspects of the present invention, cells are administered to the patient in need of a transplant in sterile saline. In other aspects of the present invention, the cells are administered in Hanks Balanced Salt Solution (HBSS), Isolyte S, pH 7.4 or other such fluids chosen from 5% dextrose solution, 0.9% sodium chloride, or a mixture of 5% dextrose and 0.9% sodium chloride. Other examples of diluents are chosen from lactated Ringer's injection, lactated Ringer's plus 5% dextrose injection, Normosol-M and 5% dextrose, and acylated Ringer's injection. Still other approaches may also be used, including the use of serum free cellular media. Systemic administration of the cells to the patient may be preferred in certain indications; whereas, direct administration at the site of or in proximity to the diseased and/or damaged tissue may be preferred in other indications, as determined by the pharmaceutical presentation and as determined by those skilled in the art.

Pharmaceutical compositions according to the present invention preferably comprise an effective number of HUCBCs within the range of about 1.0×10⁴ cells to about 1.0×10¹⁴ cells, more preferably about 1×10⁵ to about 1×10¹³ cells, even more preferably about 2×10⁵ to about 8×10¹² cells generally in suspension, optionally in combination with a pharmaceutically acceptable carrier, additives, adjuncts or excipients, as appropriate.

Preferably the umbilical cord blood cells are administered with a blood brain barrier permeabilizer. In one embodiment, the cells are combined with the permeabilizer prior to administration into the patient. In another embodiment, the cells are administered separately to the patient from the permeabilizer. Optionally, if the cells are administered separately from the permeabilizer, there is necessarily a temporal separation in the administration of the cells and the permeabilizer. The temporal separation may range from less than a minute to hours or days. The determination of the optimal timing and order of administration is determinable by one of skilled in the art.

In one embodiment, HUCBCs are administered with cyclosporine or another anti-rejection compound.

All data in the following examples were analyzed using analysis of variance (ANOVA). Post-hoc analysis was performed using the Newman-Keuls test, and the level of significance is provided where pertinent. If homogeneity of variance showed significance, the Mann-Whitney post-hoc analysis was also used. Simple linear regression was used to determine a correlation between behavior and infarct volume.

Throughout this application, various publications are referenced. The disclosures of all of these publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

### EXAMPLES

### Example 1

Permanent Middle Cerebral Artery Occlusion (MCAO or CVA). Sixty-three Sprague Dawley rats (200-250 g) were anesthetized with isoflurane (2-5% in O₂ at 2 L/min). All animals were placed on a heating pad. The right common carotid, external carotid, internal carotid and pterygopalantine arteries were isolated using blunt dissection. The external carotid was ligated, cut and an embolus made of nylon thread (25 mm long) was inserted through it. Once in place, the embolus was tied permanently, and the skin was closed.

Cell Preparation and Transplantation. The HUCBCs (Cambrex Corp, East Rutherford, N.J.) were thawed at 37° C. in Isolyte balanced electrolyte solution with a pH of 7.4. The cells were washed and centrifuged three times (1,000 rpm for 10 min). Viability was determined using the trypan blue exclusion method. Cell concentration was adjusted to 10⁶ in 500 μL, the dose that was used. The rats were randomly assigned to one of seven HUCBC transplantation groups by the time of implantation after surgery: 3 hr, 24 hr, 48 hr, 72 hr, 7 days, 1 month and MCAO only. The rats were anesthetized with 2-5% isoflurane in O₂, the penile vein was exposed, and a 31-gauge needle was inserted into the lumen of the vein for cell delivery. All animals were injected with the immunosuppressant cyclosporine A (10 mg/kg ip) at the time of the transplant and repeated for daily until sacrifice.

Behavioral Testing. All rats were tested on a series of behavioral measures (two are described here) prior to MCAO, providing a baseline measure to which behavior at two weeks and one month post-transplant were compared.

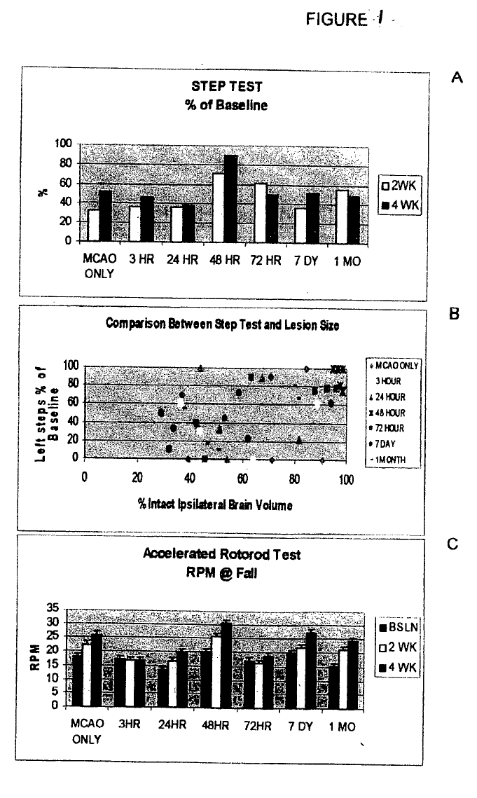

Step Test. Rats were held at a 75° angle to the tabletop with one forepaw placed on a table. They were dragged one meter in the direction of their placed paw, and the number of steps taken was recorded. Both the right and left paws were tested in random order. As the animal is moved forward along the surface, it reflexively moves its forelimb as if stepping. The number of stepping movements over a distance of 100 cm was recorded. In normal animals, the left and right forelimb steps do not differ significantly. In FIG. 1A, animals receiving ipsilateral transplants 48 hr after MCAO took significantly more steps with their left paw (the one affected by MCAO) than all other transplant groups: MCAO alone (U=0, p<0.01), 3 hr (U=1.5, p<0.01), 24 hr (U=5.5, p<0.01), 72 hr (U=3, p<0.01), 7 d (U=6, p<0.01), and 1 mo (U=3, p<0.01), using the ANOVA followed by Mann-Whitney analysis. FIG. 1B indicates that there is an inverse relationship between the number of steps made on the rat's affected limb and the infarct volume on the ipsilateral side (r²=0.174, p<0.01).

Accelerated Rotorod Test. Rats were placed on a revolving rod that increased in speed from 0-40 rpm over the course of 3 min. The more impaired that one side or another is (due to CVA), the shorter is the time on the rod and the fewer are the recorded rpm. The time and rpm at which the rat fell off the rod were recorded. FIG. 1C shows that rats that received a transplant at 48 hr following MCAO had significantly greater motor improvement than MCAO alone controls (W=2.88, p<0.05), or 3 hr (W=7.89, p<0.01), 24 hr (W=5.81, p<0.01) and 72 hr (W=4.41, p<0.01) transplant groups, using Newman-Keuls analysis.

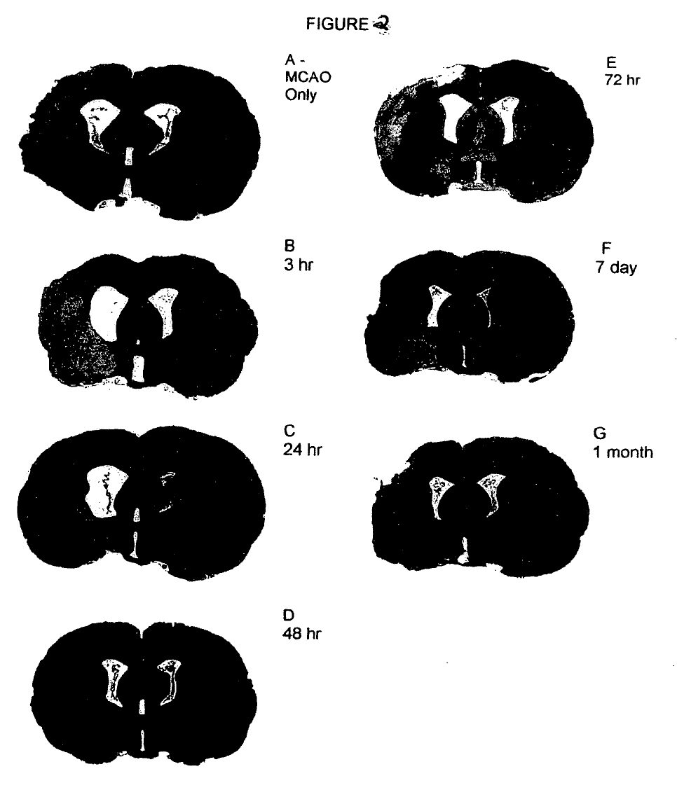

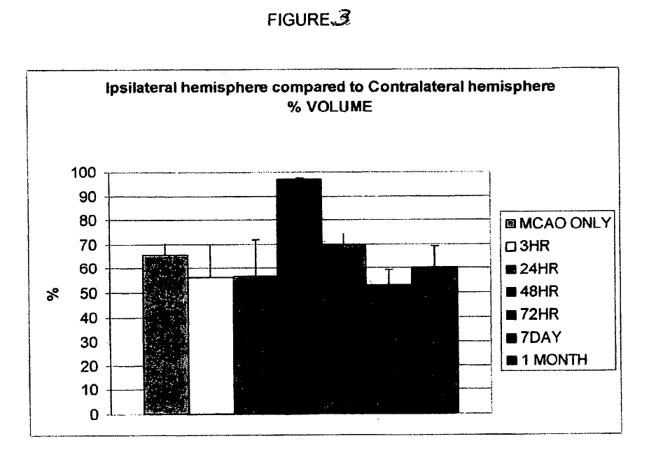

Histology and Immunohistochemistry. Following their one-month behavioral tests, rats were transcardially perfused with 4% formaldehyde in 0.1 M phosphate buffer. Their brains and other organs (heart, lungs, spleen, liver, kidneys, thymus and bone marrow) were removed, fixed for 24 hr and then cryopreserved in 30% sucrose prior to sectioning at 30 μm intervals using a cryostat (Mikron Instruments, San Marcos, Calif.). Nissl staining with thionin was used to determine the extent of infarction after MCAO at each of the seven post-MCAO time points. Sections were examined at six levels beginning 0.3 mm anterior to bregma and then at 1 mm intervals to 3.3 mm posterior to the bregma. The slide-mounted sections were hydrated, stained for Nissl substances with thionin for 90 sec, and then rinsed in xylene and cover-slipped with Permount mounting solution. FIG. 2, from left to right, shows staining for MCAO only (FIG. 2A), 3 hr (FIG. 2B), 24 hr (FIG. 2C), 48 hr (FIG. 2D), 72 hr (FIG. 2E), 7 d (FIG. 2F), and 1 mo (FIG. 2G). The results of dividing the uninfarcted volume of the ipsilateral side of the brain by the volume of the contralateral side are reported in percents for each time period (FIG. 3). Significantly greater loss of cells in the striatum and cortex on the ipsilateral side versus the contralateral side, except at the 48-hr transplant time point, were apparent. The 48-hr HUCBC treatment produced a result near that of 100% of normal (p=0.024); whereas, infarct volumes were greater (and intact tissue smaller when HUCBCs were administered at different times. These data further support that the optimal therapeutic effectiveness of HUCBCs is at about 48 hr after injury, when pathophysiological responses of the test animals achieved maximal chemo-attractant capacity for HUCBCs.

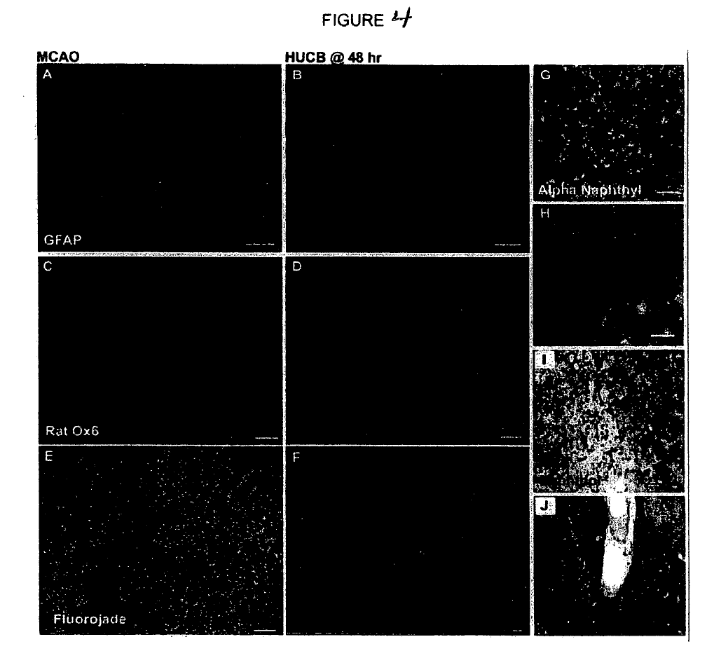

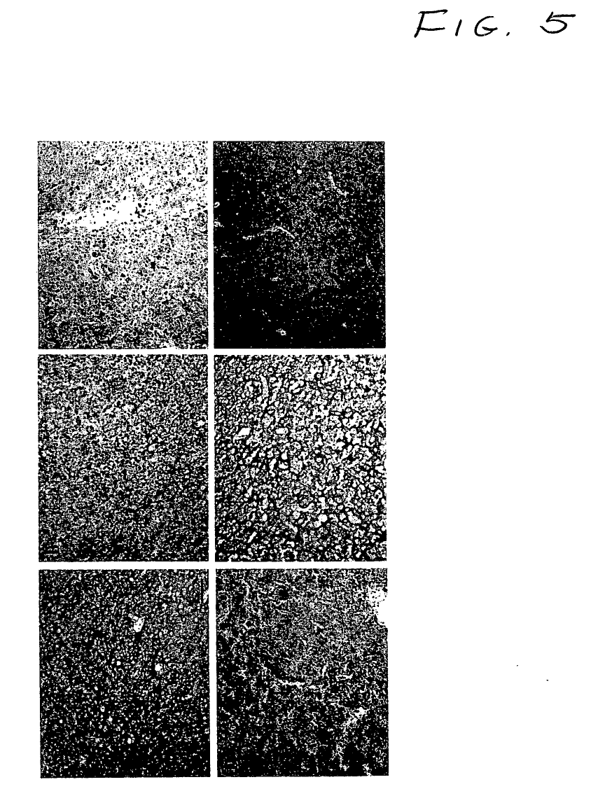

Naphthol AS-D chloroacetate esterase labels granulocytes, and α-naphthol acetate esterase labels monocytes. Naphthol AS-D chloroacetate (Sigma Aldrich, St. Louis, Mo.) and other solutions were prepared per kit instructions. Tissue slides were incubated for 15 min in prewarmed naphthol solution (40°) and were protected from light. The slides were then rinsed in deionized water and counterstained with hematoxylin solution for 2 min, rinsed with tap water and allowed to air dry prior to cover-slipping with glycerol. α-Naphthyl acetate esterase (Sigma-Aldrich) solutions were prepared per kit instructions. Tissue slides were incubated in prewarmed α-naphthyl solution for 30 min and protected from light. The slides were rinsed with deionized water, counterstained for 2 min with hematoxylin solution, rinsed with tap water and allowed to air dry before being cover-slipped with glycerol. At one month post-stroke, MCAO-only controls (FIG. 4G, scale bar=100 μm) exhibited more intense staining of monocytes than 48 hr HUCBC-treated rats (FIG. 4H, scale bar=100 μm). Greater numbers of neutrophils were found in MCAO-only controls (FIG. 41, scale bar=50 μm) than in 48 hr HUCBC-treated rats (FIG. 4H, scale bar=50 μm). Neutrophils in the 48 hr-treated rats were largely confined to vessels.

The glial fibrillary acidic protein test (GFAP, Dako, Carpinteria, Calif.) started with the slides being incubated in polyclonal primary GFAP antibody (1:750) overnight at 4° C. Slides were then rinsed and incubated in rhodamine-conjugated anti-primary antibody (Molecular Probes, Eugene, Oreg., 1:200) for 2 hr at room temperature. Astrocytes were identified using antibody against GFAP. Staining was more intense in the MCAO-only control (FIG. 4A, Scale bar=50 μm), than in rats receiving HUCBCs at 48 hr (FIG. 4B, scale bar=50 μm). These data show that the 48-hr, HUCBC-treated brain had fewer inflammatory cells.

Activated microglia were identified using antibody against rat MHCII. For the rat MHCII test (Serotec, Raleigh, N.C., 1:300) slides were incubated in the monoclonal anti-MHCII antibody overnight at 4° C. The slides were then rinsed and incubated for 2 hr in fluorescein isothiocyanate (FITC)-conjugated secondary antibody (Molecular Probes, 1:200) at room temperature. Photomicrographs (FIG. 4C-4G) show that rats treated with HUCBCs 48 hr after MCAO injection (FIG. 4D, scale bar=200 μm) showed no staining in contrast to MCAO-only controls (FIG. 4C, scale bar=200 μm). These data also indicate that HUCBCs reduced microglial inflammation that otherwise caused the additional behavioral deficits discussed supra.

For the fluorojade (Histochem, Jefferson, Ariz.) test, slides were hydrated and immersed in 0.06% potassium permanganate for 15 min. The slides were rinsed in deionized water and incubated in the fluorojade solution for 30 min, rinsed again and cover-slipped. Fluorojade staining helps identify dead and dying cells. Similar to thionin (supra), intense staining is observed in MCAO only controls (FIG. 4E, scale bar=200 μm), but not 48 hr-HUCBC-treated rat (FIG. 4F, scale bar=200 μm).

### Example 2

Once treatment at approximately 48 hr was demonstrated to contribute to the greatest physiological and behavioral recovery, a group of animals was subjected to MCAO while monitored with laser Doppler to verify that the MCAO technique was consistent in its ability to produce a severe drop in cerebral blood flow. Animals in this group reproduced the findings supra of cell death and inflammation after MCAO and HUCBC transplantation and confirmed the therapeutic value of HUCBC therapy applied at the appropriate time interval.

Prior to MCAO surgery, rats were anesthetized and maintained with isoflurane (2-5% in O₂ at 2 L/min), and a small access port was drilled through the skull 1 mm posterior and 4 mm right lateral to the bregma. A fiber optic filament was placed through the access port to rest on the dura mater, with care taken to avoid disturbing the meninges and cerebral cortex, and was connected to the laser Doppler (Motor Instruments, Devon, UK) which recorded cerebral pressure changes throughout the MCAO surgery. Unlike preceding tests, these rats were not given cyclosporine during this study. The criterion for subject inclusion in the study was a severe drop from baseline pressure.

Tissues were prepared as in Example 1. However, for apoptosis detection, frozen sections were prepared and treated as per the NeuroTacs kit instructions (Trevigen, Inc., Gaithersburg, Md.). Briefly, thaw-mounted cryostat sections were incubated in NeuroPore detergent buffer for 30 min, rinsed in two changes of DNase free water for 2 min each, then immersed in Quenching solution for 5 min. Slides were washed with phosphate buffered saline (PBS) for 1 min and immersed in 1× terminal deoxynucleotidyl transferase (TdT) Labeling Buffer for 5 min. Then Labeling Reaction mix was pipetted onto each section and incubated for 60 min, after which the slides were immersed in Stop Buffer for 5 min. The sections were then washed twice with PBS for 2 min each and then Streptavidin HRP (Streptavidin horseradish peroxidase) solution was pipetted onto each section and incubated for 10 min and again washed in 2 changes of phosphate-buffered saline (PBS) for 2 min each. The slides were then immersed in DAB solution for 5 min, washed in two changes of distilled water for 2 min each, and then immersed in blue counterstain for 1 min. The sections were then rinsed in tap water and ammonium water, and then were dehydrated and cover-slipped with Permount solution. Positive and negative controls were prepared on each slide.

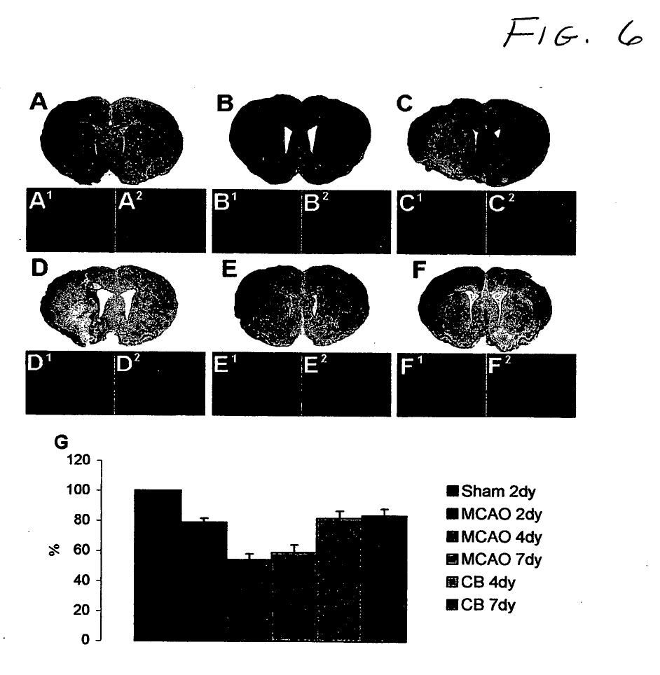

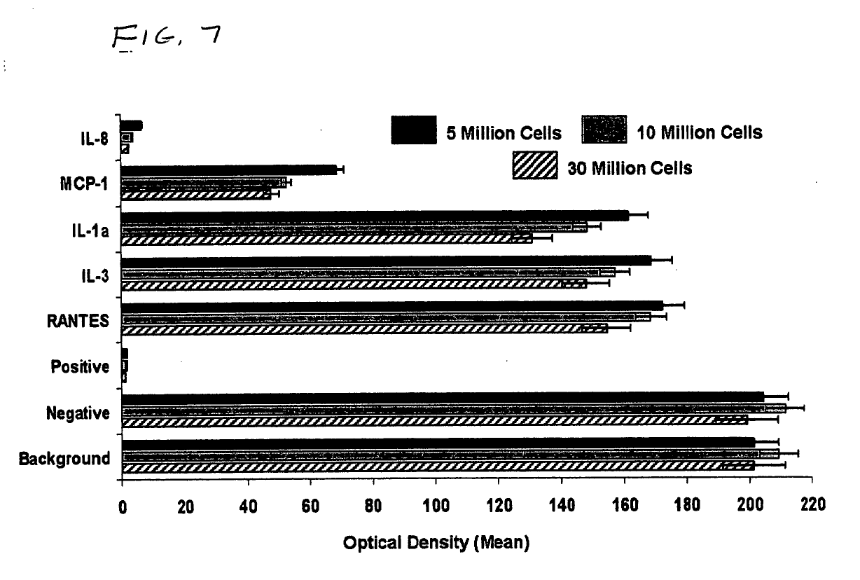

We observed TdT deoxyuridine nicked end labeling (TUNEL)-positive cells indicative of apoptotic death in the MCAO lateral striatum (ischemic core) over the course of 7 d following MCAO. Apoptotic cell death in the core was reversed by HUCBC transplants at 48 hr transplantation. HUCBC transplantation at 48 hr inhibited apoptosis, possibly through expression of anti-apoptotic genes such as Bcl-2 or Bcl-X_{L}. Sham-operated rats served as controls (FIG. 5A). Cells undergoing apoptosis in the core of the infarct reached their maximum about 48 hr after MCAO (FIG. 5B); however, in untreated MCAO brains, cells continued to undergo apoptosis with TUNEL positive cells still being observed at 4 d (FIG. 5C) and 7 d (FIG. 5D) after MCAO. When HUCBC treatment was given at 48 hr, no apoptotic cells were observed at 4 and 7 d after MCAO (FIGS. 5E and 5F). Rats treated with HUCBC at 48 hr after MCAO resembled sham-operated rats upon examination at 4 and 7 d—with little astrocytic or microglial activation (FIGS. 6A1-6F1 and 6A2-6F2, respectively). Most of these rats had nearly intact cytoarchitecture, similar to our recent observations (FIGS. 6E-6F). In contrast, infarct volume was minimal at 2 days after surgery in the MCAO-only rats, establishing that infarct expansion was greatest at this time point and but data showed that infarct volume continued to expand for at least 4 days if not longer in this CVA model. (FIG. 6B-6D). These data showed that a much larger percentage of cells can still be rescued at later time points than previously thought possible.

### Example 3

The cytokine release from the ischemic areas after MCAO at different time points and from HUCBCs themselves were investigated. Both monocytes-chemoattractant protein 1, MCP-1, and growth-related oncogene/cytokine-induced neutrophil chemoattractant-1, GRO/CINC-1 (the rat equivalent of human IL-8), were elevated in rat ischemic tissue extract in the cortex, striatum and hippocampus. Results of these ELISAs showed a time-dependent pattern similar to that observed with the migration data (Newman et al., 2003a, ibid.). In addition, our initial cytokine arrays of the HUCBCs showed that the mononuclear fractions of this cell population released MCP-1, IL-8, epithelial cell-derived neutrophil activating protein (ENA-78), and macrophage derived chemokine (MDC).

The mononuclear fraction of the HUCBCs was obtained from Saneron CCEL Therapeutics, Inc. (Oldsmar, Fla.). Frozen samples were thawed in 10 mL of DMEM (Gibco), supplemented with 5% fetal bone serum (FBS, Gibco) and gentamicin (50 μg/mL, Sigma), or with Ex Vivo 10 media with gentamicin (50 μg/mL, Sigma). After centrifugation for 10 min at 200 rpm, the supernatant was removed and the cells were resuspended in 1 mL of the same medium. The viability of all samples ranged from 73% to 95% as determined by the ability to exclude trypan blue dye. Cells were then cultured for 3 to 14 d for cytokine arrays and ELISA assays; cells cultured in Ex Vivo 10 media were further stimulated with IL-3, TPO or both.

MCAO surgery was conducted as described supra. Sham rats received the same surgery, except that the middle cerebral artery was not blocked. The animals were sacrificed at 4, 6, 24, 48 and 72 hr, and 1 wk after ischemic injury. The brains were removed within two min; the ipsilateral and contralateral sides were dissected, then rapidly frozen, and stored at −80° C. Ischemia tissue extracts and normal rate tissue extracts from the same brain areas were used in both cytokine arrays and ELISAs.

Preparation of Brain Tissue Extracts: Frozen tissue sections were kept on ice; and the striatum, hippocampus and cortex were dissected from the ipsilateral and contralateral sides to the occlusion in MCAO animals and from the left and right side of the brains of sham-surgery and normal rats. The sections were then homogenized in a clear medium (150 mg/mL of DMEM). The homogenates were centrifuged. The resulting supernatants were extracted and then filtered through a 0.22μ filter. The filtered extracts were then frozen.

Protein BCA Assays and Standard Curves: BCA Protein Assays (Promega) for ischemic tissue extracts and for conditioned media from HUCB cultured cells were performed twice, and unknown samples and standards were performed in triplicate (three wells per assay for six data points per assay). These extracts were pipetted directly into the bottom well of a 96-well plate. Standard curves were run in the same 96-well plate at the time of assay and for determining sensitivity of the plate reader (Bio-Tech, Inc.).

Human Cytokine Arrays: The therapeutic benefit of the HUCBCs may be through production of cytokines or chemokines at the site of injury. In order to address this, we used human cytokine arrays to establish whether the HUCBCs secreted these proteins. To determine the cytokines that HUCBCs released during culture, we used a TranSignal human cytokine antibody array (Panomics, Inc., Redwood City, Calif.), which simultaneously profiles either 23 (Array 1.0) or 42 (Array 3.0) cytokines/assay at the protein level. First, we examined whether cytokine production increased as a function of seeding density. The HUCBC were cultured at concentrations of 5, 10, or 30×10⁶ per 5 mL of serum free DMEM with Gentamicin (50 μg/mL) for 3 d. The conditioned medium from all three concentrations expressed the same 5 cytokines: IL-8, MCP-1, IL-1a, IL-3, and RANTES (regulated on activation, normal T-cell expressed and secreted), which were all significantly more dense than those in controls (FIG. 7). There was a progressive increase in intensity of these cytokines that corresponded to the increase in HUCBC concentration.

Next we examined if HUCBC production of cytokines was altered by stimulation with factors known to stimulate hematopoietic cells, namely, IL-3 and thrombopoietin (TPO). The mononuclear fractions of HUCBCs were thawed and cultured (10⁷ cells/5 mL) as follows:
1. Ex Vivo 10 medium (Cambrex) with gentamicin (50 μg/mL, Sigma) for 1, 5, or 12 d.
2. Ex Vivo 10 medium (Cambrex) with gentamicin (50 μg/mL, Sigma) for 4 d followed by 5 d with 5 ng/mL IL-3 in the medium. Medium was changed to control medium on d 9, and cultures were processed after 12 DIV.
3. Ex Vivo 10 media (Cambrex) with gentamicin (50 μg/mL, Sigma) for 4 d followed by 5 d with 25 ng/mL TPO in the medium. Medium was changed to control media on d 9, and cultures were processed after 12 DIV.
4. Ex Vivo 10 medium (Cambrex) with gentamicin (50 μg/mL, Sigma) for 1, 5, or 12 d. The HUCBCs were cultured at 5×10⁷ cells/5 mL.

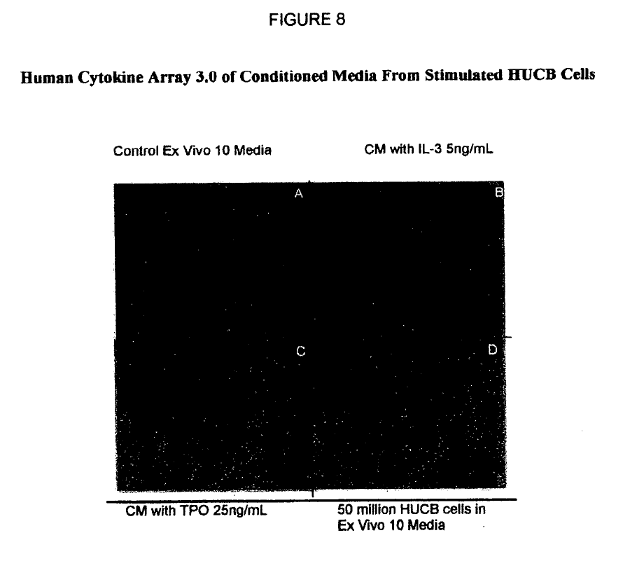

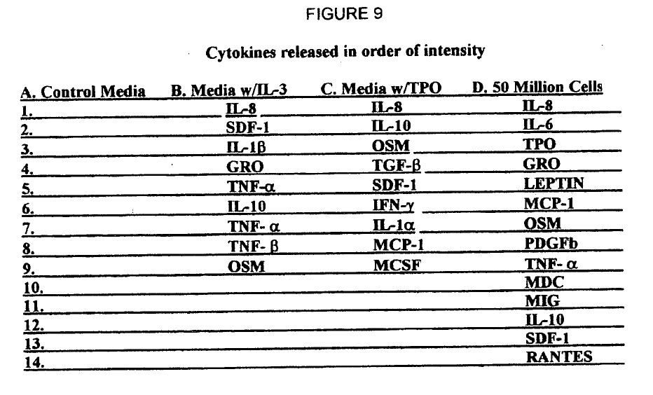

The medium from these cultures was harvested and incubated with the array membrane to allow cytokine binding to immobilized antibodies spotted on the arrays. The assays were performed according to the manufacturer's instructions. Results were visualized using streptavidin-HRP and chemiluminescence detection on x-ray film. FIGS. 8A-8D are radiographs of the 42-cytokine arrays. FIG. 8A shows a membrane exposed to Ex Vivo 10 medium alone; in this case, positive controls are visible, but few cytokines bound to the membrane. FIG. 8B shows the results with conditioned medium from HUCBCs treated with IL-3 (5 ng/mL), which caused the release of many cytokines. FIG. 8C shows the results with conditioned medium obtained from HUCBCs treated with thrombopoietin (25 ng/mL), displaying a somewhat different profile of protein release. The most cytokines were recorded with 5×10⁷ million HUCBCs in Ex Vivo 10 Medium alone. These findings are summarized in FIG. 9, which lists the particular cytokines released as functions of culture conditions and intensity (most intense listed first). When compared to serum-free media alone, HUCBCs first cultured in DMEM with 10% FBS for four days and then DMEM with no FBS for 6 days released (in order of intensity) IL-8, MCP-1 ENA-78 and MDC (FIGS. 10A and 10B).

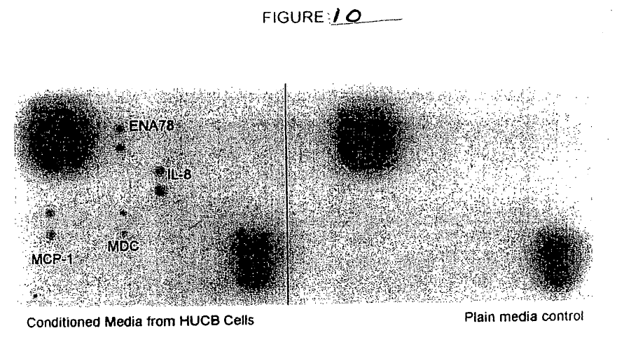

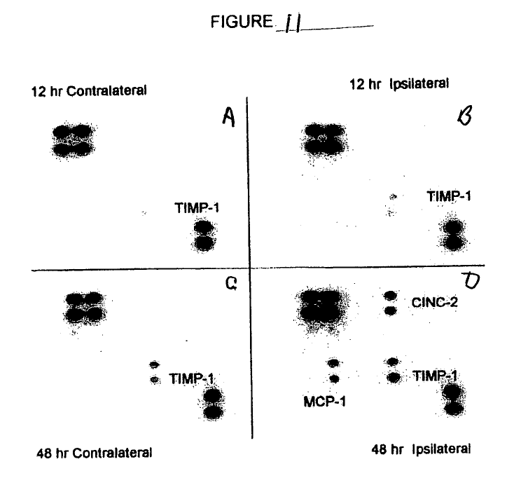

Rat cytokine array. To determine cytokines in striatal ischemic tissue extracts, 500 μL of supernatant was diluted with 500 μL of DMEM. Such 1:1 dilution was performed to assure that the results for the ischemic extracts were in the range of the cytokine array detection. The tissue extracts were obtained according to the method discussed above. The rat cytokine array with 19 cytokine antibodies was performed according to the manufacturer's instructions. Results were visualized by using streptavidin-HRP and chemiluminescence detection on radiographic film. Referring now to FIGS. 11 and 12, in all conditions, except the two controls and the one-week contralateral side, tissue inhibitor of metalloproteinase-1 (TIMP-1) was shown to be present. The ischemic tissue extracts at 48 hours showed the presence of MCP-1, cytokine induced neutrophil chemoattractant-2 (CINC-2), the former of greater intensity. At the one-week time point, the results were similar, except β-nerve growth factor (β-NGF) was also present, but less intense than the other cytokines present.

ELISA for Rat GRO/CINC-1 and MCP-1. MCAO rat tissue extracts were prepared as previously described and stored at −80° C. until needed. Brain samples were taken from rats at 4, 6, 12, 24, 48 and 72 hr and at 1 wk after CVA. The controls were the contralateral side of the same rat, sham surgery and normal rats.

GRO/CINC-1 is closely related to human IL-8, which is lacking in rats. For the rat GRO/CINC-1 ELISA, 100 μL of MCAO and control tissue extracts were incubated in 96-well plates for one hr with immobilized polyclonal antibodies to rat GRO/CINC-1, after which bound cytokine was incubated with appropriate labeled antibody and substrate solutions. The standard curve was performed according to the manufacturer's instructions (TiterZyme EIA, Assay Designs, Inc., Ann Arbor, Mich.) with a range of 0-300 μg/mL. The plates were then read in the BioTech plate reader set to absorbance at 450 nm. There was no significant difference in the sham surgery rats at any of the time points (data not show). GRO/CINC-1, as well as IL-8, is a chemoattractant for neutrophils, causing neutrophil infiltration into inflammatory sites. Overall, there was a trend that resulted in a higher level of GRO/CINC-1 in the ipsilateral side of the ischemic tissue extract in striatum (FIG. 13A) and hippocampus (FIG. 13B). This was also true for the surrounding cortex (data not shown). FIGS. 13A and 13B show the results for GRO/CINC-1 in striatal and hippocampal extracts, respectively. GRO/CINC-1 is present in pg/mL amounts in rat ischemic tissue extracts.

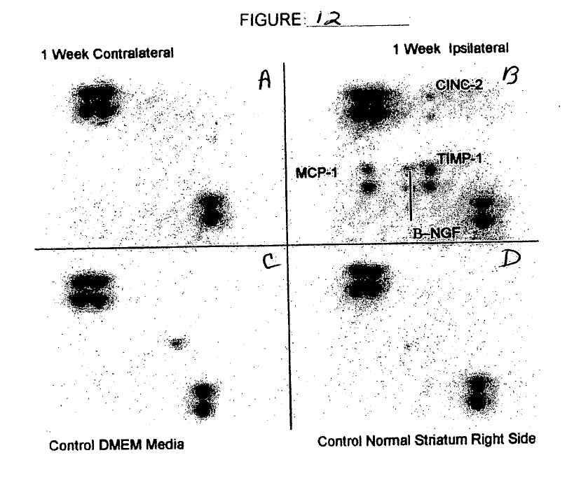

For the rat MCP-1 ELISA, 50 μL of MCAO and control tissue extracts were incubated in a 96-well strip plate for 1 hr with immobilized antibody to rat MCP-1 after which bound MCP-1 was incubated with appropriately labeled antibody and substrate solutions. The standard curve was performed according to manufacturer's instructions (Rat MCP-1 ELISA Kit, Pierce Endogen, Rockford, Ill.) with a range of 0-1500 pg/mL. Plates were then read in the BioTech plate reader set to 450 nm absorbance. FIGS. 14A and 14B show the levels of MCP-1 in rat striatal and hippocampal tissue extracts, respectively. The data show an overall trend that resulted in a higher level of MCP-1 on the ischemic side in both the striatum and hippocampus. The surrounding cortex was also examined with similar results (data not shown). This was the first study of MCP-1 over time in the focal model of CVA. In addition, MCP-1 has also been reported in serum during the first 24 hr after myocardial infarction. Administration of HUCBCs at 2-24 hr are expected to optimize the effects of HUCBCs in AMI. This chemokine attracts not only monocytes, but also activated T-cells and may also activate macrophages.

Human Cvtokine Array. The conditioned medium from HUCBCs was analyzed for 23 different cytokines. Ten million (10⁷) HUCBCs were cultured in DMEM with 10% FBS for four days. Medium was then switched to DMEM with no FBS for an additional six days, for a total of 10 DIV. HUCBCs, when cultured in serum-free medium, release (in order of intensity) IL-8, MCP-1, ENA-1 and MDC, when compared to serum-free medium only.

### Example 4

To examine whether MCP-1, IL-3 or TPO were chemoattractant(s) for HUCBCs, migration assays were performed. MCP-1, IL-3 and TPO were selected as chemoattractants because MCP-1 has been found in rat ischemic striatal tissue and IL-3 and TPO are often added to culture medium for maintaining long-term cultures and enhancing progenitor cell proliferation.

HUCBCs were prepared as described above and cultured for 24 hr before use. The 96-Chemotx chambers (NeuroProbe, Gaithersburg, Md.) were used for the migration assays. The chamber is a 96-well plate consisting of bottom wells to hold the unknowns or chemoattractant and a top plate, which is a polycarbonate membrane with 5 μm pore size. The chemoattractants used in this study were human recombinant MCP-1 (5, 10, 20 and 30 ng/mL; Endogen, Woburn, Mass.), IL-3 (1, 5 and 10 ng/mL), TPO (10, 25 and 40 ng/mL), and stromal cell-derived factor-1 (SCF-1; 100 ng/mL); positive control (SDF-1; Serologicals, Norcross, Ga.), along with the serum-free DMEM and Ex Vivo 10. All were pipetted (300 μL) directly into the bottom wells, in triplicate, after which the top plate was securely attached. The HUCBCs were pipetted directly into each top well at a concentration of 62,000 cells per 25 μL. The migration chambers were then placed in a cell culture incubator at 37° C. with 5% CO₂ for 4 hr. The top plates were removed and the bottom plates were then centrifuged (300 rpm for 10 min) to force cells to the bottom. Half the medium (150 μL) was then removed and a cell viability assay (CellTiter-Glo Kit, Promega), which is based on the ability of live cells to incorporate adenosine triphosphate (ATP), was used to determine the numbers of cells that had migrated. The migration plate was read in an automated plate reader (BioTek) set to the appropriate luminescence.

Here, HUCBCs strongly migrated to MCP-1 (FIG. 15), which has been found in rat ischemic tissue and could explain the success of Example 1. Significantly more HUCBCs migrated to MCP-1, IL-3 and TPO when compared to controls (DMEM and PBS). Astrocytes and microglia secrete more MCP-1 in medium than do neurons (data not shown). Normotoxic tissues do not attract HUCBCs nearly as well (data not shown). Hence, areas of excess astrocytes and microcytes attract HUCBC in preference to normotoxic tissues.

### Example 5

In determining the time course of HUCBC migration into brain after a stroke, an ex vivo experimental approach was employed. Male Sprague Dawley rats under a permanent middle cerebral artery occlusion (MCAO); a nylon filament was threaded through the internal carotid artery to the origin of the MCA and permanently ligated in place. At 4, 6, 12, 24, 48, 72 hrs and 1 week after ischemia the animals were euthanized and the brains removed within two min, immediately flash-frozen and then stored at −80° C. until needed. The frozen brains were semi-thawed and kept cold. The striatum, hippocampus, and the cortex were dissected from the sides both ipsilateral and contralateral to the occlusion. The same dissections were performed for sham surgery and normal rats. Tissue from each of the conditions were pooled, and kept on ice in clear Dulbecco's modified Eagle's medium (DMEM, Invitrogen, Carlsbad, Calif.). Tissue (150 mg of tissue per 1 mL of medium) was homogenized. The homogenates were centrifuged (4000 rpm for 20 min), the tissue extracts (supernatant) collected, filtered (0.22 μm, Millipore, Bedford, Mass.), and stored at −80° C. until used.

For the migration assay, the tissue extracts (supernatant) of each brain area (striatum, hippocampus, and cortex) from both the ipsilateral and contralateral sides and for each time period (4, 6, 12, 24, 48, 72 hours, or 1 week after stroke) were pooled together per condition. The 96-Chemotx® Chambers (Neuro Probe, Gaithersburg) were used for these migration assays. The chamber is a 96-well plate consisting of bottom wells that hold the unknowns or chemoattractant and a top plate, which has a polycarbonate membrane with 5 μm pore size. Either 300 μL of the tissue extracts (unknowns), standards, or controls were pipetted into the bottom wells, in triplicate, after which the top plate was securely attached to the bottom plate.

The cryopreserved HUCBCs were thawed and transferred into clear DMEM with 5% FBS and 1 μL/1 mL of Gentamicin (Sigma, St. Louis). Cells were then centrifuged at 400 g for 15 minutes, the supernatant was removed, and the cells resuspended in 1 mL of media. Cord blood cells were then plated in low adherence 6 well culture dishes (Corning, Corning, N.Y.) for 24 hrs in a water jacket incubator set at 37° C. and with 5% CO₂ After 24 hrs cells were lifted by gentle pipetting, placed in a 15 mL tube, centrifuged, resuspended in 1 mL of media without FBS, and viability assessed using the trypan blue dye exclusion method. Only HUCBCs with 80% or greater viability were used and cell concentration was adjusted to 62,000 cells/25 μL of media. The cells were then pipetted directly into the top well at a concentration of 62,000 cells per 25 μL. The migration chambers were then placed in a water-jacket incubator at 37° C. with 5% CO₂ for 4 hours. The top plates were removed and the bottom plates were then centrifuged (300 g for 10 minutes) so that all migrating cells would be forced to the bottom. Half the media (150 μL) was then removed and a cell viability assay (CellTiter-Glo Kit, Promega), which is based on the incorporation of adenosine 5′ triphosphate (ATP) into live cells, was used to determine the number of HUCBC that had migrated to the bottom well. Stromal derived factor 1 (SDF-1) was used as a positive control and media as a negative control.

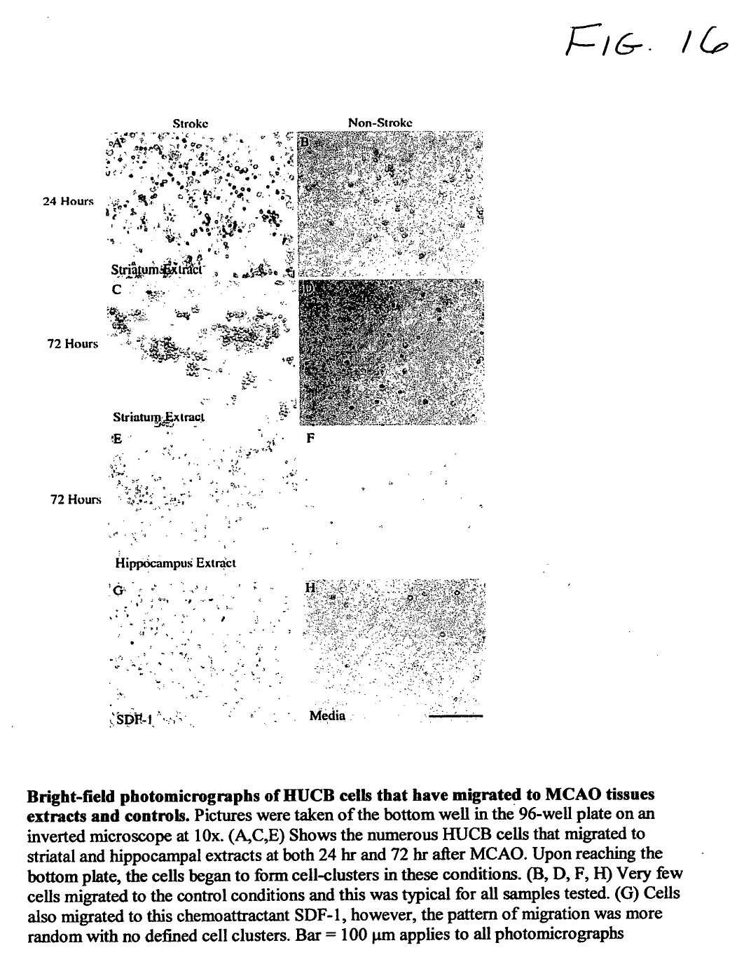

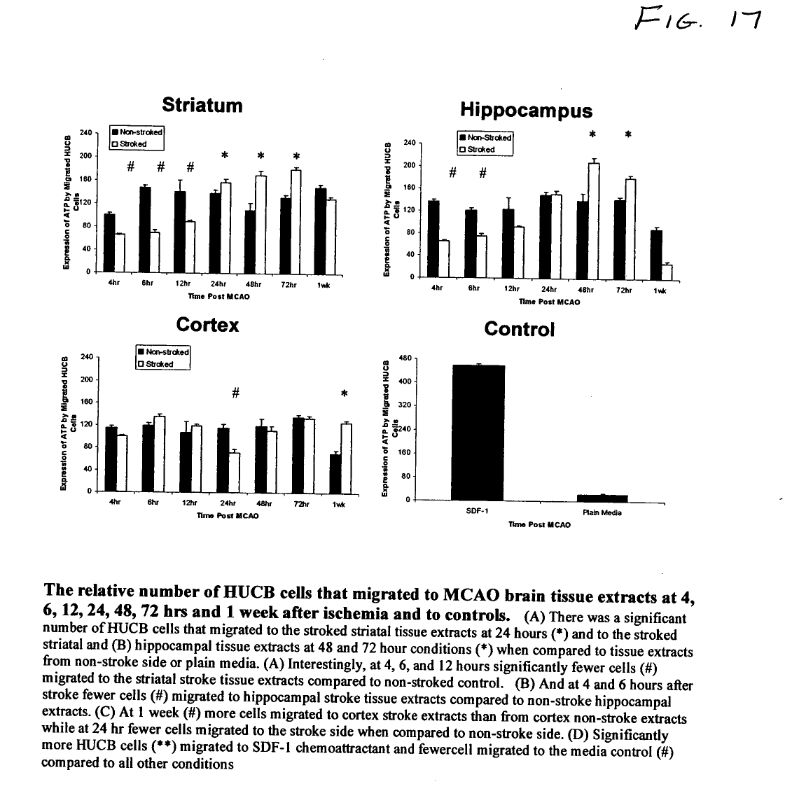

Under these conditions, HUCBC migrated more toward extracts of the infarcted (ipsilateral) brain than to normal (contralateral) brain (FIG. 16). Prior to 24 hours, migration toward the extract of stroked striatum was inhibited, but from 24-72 hours significantly more HUCBC migrated toward the stroke extract compared to normal extract (FIG. 17). Similar results were observed with hippocampal extracts, although only at 48 and 72 hours was there greater migration toward the stroke extract than the normal extract.

Discussion. The cytokine arrays have helped us to examine the complex responses to infarction and the action of HUCBCs in response to the subsequent pathophysiological processes. These studies demonstrate the significant signal interaction that occurs between HUCBCs and ischemic tissue in order to produce a variety of cytokines and chemokines, the profiles of which change according to ambient conditions. HUCBC extensively produced both IL-8 and MCP-1, which are considered the first line of defense in the inflammatory reaction. IL-8, or the rat equivalent GRO/CINC-1, has been shown to be elevated from 24 hr to 72 hr after CVA when compared to non-CVA tissues. IL-8 also is elevated in a number of human injuries and diseases, such as 1) serum of patients with multiple sclerosis, 2) coronary artery disease, 3) traumatic brain injury, and 4) CVA patients. In addition, IL-8 from cord blood alone or together with other cytokines is being used as a determinant for neonatal sepsis.

TNF-α and IL-1 have been implicated as the cytokines responsible for stimulating the release of IL-8 and MCP-1. Recently the chemoattraction of neutrophils to IL-8 was shown to be dependent on CINC-1 produced from mast cells. This discovery helps explain the migration of HUCBCs to ischemic tissue. Both neutrophils and mast cells are in the heterogeneous population of HUCBCs and, depending on the culturing conditions, may be preserved for long periods. In addition, ischemic tissue extracts, previously shown to express CINC-1 and CINC-3, have revealed the presence of IL-8 in every HUCBC culture condition. These lines of evidence indicate that HUCBCs are partially attracted to ischemic tissue due to its content of CINC-1, the variety of and the interaction of cells within the cord blood (including neutrophils and mast cells), and the production of IL-8 from these cells.

MCP-1 is a β-chemokine that attracts monocytes for a 48-hr period after interaction of antigen and sensitized lymphocytes. Following Ex Vivo 10 conditions for 1 and 12 days in culture, the presence of IL-6 in HUCBCs was more intense than that of MCP-1. In the IL-3 stimulated condition, the presence of the chemoattractant was not nearly as intense when compared to the other conditions.

Surprisingly, HUCBCs when cultured in a hematopoietic medium (Ex Vivo 10 medium) at 5 and 12 DIV and with the addition of IL-3 or TPO, SDF-1 production was induced, which was not seen in any of the conditioned medium from cord blood cells cultured in DMEM. SDF, a multifaceted chemoattractant induces the homing and mobilization of hematopoietic stem cells, especially to bone marrow, enhances cell survival alone or with other cytokines, potentiates angiogenesis and potentiates the migration of cord blood cells. SDF also induces IL-8 from cord blood-derived human mast cells. However, in this study, HUCBCs that were in culture conditioned without SDF-1 also induced IL-8, which indicates that the presence of SDF-1 is not essential to induce for IL-8.

This invention has been described in an illustrative manner, and it is to be understood that the terminology used is intended to be in the nature of description, rather than limitation. Obviously, many modifications and variations of the present invention are possible in light of the above teachings and one of ordinary skill in the art, in light of this teaching, can generate additional embodiments and modifications without departing from the spirit of or exceeding the scope of the claims of this invention. Therefore, it is to be understood that within the scope of the appended claims, the invention may be practiced otherwise than as specifically described. Accordingly, it is to be understood that the drawings and descriptions herein are proffered by way of example to facilitate comprehension of the invention and should not be construed to limit the scope thereof.

1. A method for repairing animal tissue damage due to an inflammatory reaction in an animal, the method comprising
a. providing umbilical cord blood cells (UCBCs) in a pharmaceutically acceptable form; and
b. administering a sufficient dose of UCBC at an optimal time, thereby reducing the injury from the inflammatory reaction.

2. The method of claim 1 wherein the optimal time is 48 hours.

3. The method of claim 1 wherein the optimal time is more than 24 hours.

4. The method of claim 3, wherein the optimal time is more than about 26 hours, about 28 hours, about 30 hours, about 32 hours, about 35 hrs, about 38 hours, about 40 hours, about 42 hours, about 44 and about 46 hours.

5. The method of claim 1 wherein the optimal time is less than 72 hours.

6. The method of claim 5, wherein theoptimal time is less than about 70 hours, about 68 hours, about 65 hours, about 62 hours, about 60 hours, about 58 hours, about 55 hours, about 52 hours and about 50 hours.

7. The method of claim 1 wherein the optimal time is between about 26 hours and about 70 hours, between about 28 hours and about 68 hours, between about 30 and 65 hours, between about 32 hours and about 62 hours, between about 32 hours and 60 hours, between about 35 hours and about 58 hours, between about 38 hours and about 55 hours, between about 40 hours and about 52 hours, between about 42 hours and about 50 hours, or between about 45 hours and about 48 hours.

8. The method of claim 1 whereby the UBCBs are administered by a parenteral route.

9. The method of claim 8 wherein the UCBCs are administered intravenously, intraarterially, intramuscularly, subcutaneously, transdermally, intratracheally, intraperitoneally or into spinal fluid.

10. The method of claim 1 whereby the UCBCs are administered to the site of inflammation or injury.

11. The method of claim 10 wherein the UCBCs are administered into an ischemic area.

12. The method of claim 11 wherein the UCBCs are administered into ischemic tissue in the brain.

13. The method of claim 1 wherein the UCBCs are administered in an amount sufficient to treat the particular site and size of the inflammation or injury.

14. The method of claim 13, wherein the UCBCs are administered in a sufficient amount, factoring in the route of administration.

15. A method of treating a patient's Multiple Sclerosis after a flare-up, comprising administering to the patient within 48 hours of a flare-up a sufficient quantity of human umbilical cord blood cells (HUCBCs) into the spinal fluid or bloodstream.

16. The method of claim 15, wherein the method further comprises delivering the HUCBCs into the spinal fluid by way of an implanted pump.

17. A method for treating acute central nervous system inflammation in a patient, the method comprising administering a sufficient quantity of HUCBC in a physiologically compatible solution to an individual suffering from an acute central nervous system inflammation.

18. The method of claim 17 wherein the quantity of HUCBCs administered is in the range of about 10⁵ to about 10¹³ and is administered at about 48 hours.

19. The method of claim 17 wherein the quantity of HUCBCs administered is 5×10⁶ per kilogram and is administered at about 48 hours.

20. The method of claim 17 wherein the HUCBCs are administered to a patient diagnosed with meningitis, trauma or cerebrovascular accident (CVA) within about 48 hours of onset or injury.

21. The method of claim 17 wherein CVA is thrombolic or hemorrhagic.

22. A method of treating myocardial ischemia in an individual comprising
a. providing HUCBCs in a physiological solution; and
b. administering the HUCBCs to the individual experiencing myocardial ischemia at a time that is 2-24 hrs after the onset of ischemia.

23. A method of treating bronchopulmonary distress in a neonate comprising
a. providing HUCBCs in a physiological solution; and
b. administering the HUCBCs to the individual experiencing myocardial ischemia at a time that is 2-24 hrs after the onset of ischemia.

24. A kit for determining when HUCBCs should be administered to an individual with an inflammatory condition, the kit comprising
a. at least one container containing antibodies specific for IL-8 and MCP-1;
b. directions for obtaining and preparing a tissue sample, directions for performing a test of IL-8 and MCP-1 in the sample, directions for interpreting the amounts of IL-8 and MCP-1 in the sample.

25. The kit of claim 22 wherein the kit comprises two containers, one containing antibody to IL-8 and one containing antibody to MCP-1.

26. The kit of claim 22 wherein the tissue is blood, spinal fluid, biopsy, or bronchial lavage.

27. The kit of claim 22 further comprising antibodies to TIMP-1 and β-NGF, the former being a control to MCP-1 and IL-8 and the latter indicating a later marker of inflammation.
